(19) United States
(12) Patent Application Publication
(10) Pub. No.: US 2006/0142395 A1
(43) Pub. Date: Jun. 29, 2006

(54) Modulation of intracellular calcium signaling by N-acylethanolamines

(75) Inventors: Peter Koulen, Benbrook, TX (US); Kent D. Chapman, Denton, TX (US)

Correspondence Address: CHALKER FLORES, LLP, 2711 LBJ FRWY, Suite 1036, DALLAS, TX, 75234, US

(73) Assignee: University of North Texas Health Science Center, Fort Worth, TX (US)

(21) Appl. No.: 10/840,449

(22) Filed: May 6, 2004

Related U.S. Application Data
(60) Provisional application No. 60/468,160, filed on May 6, 2003.

(51) Int. Cl. A61K 31/16

(52) U.S. Cl. 514/625

(57) ABSTRACT

The present invention includes compositions and methods for neuroprotection by modulating intracellular calcium concentrations by administering an effective amount of an N-acylethanolamine to a subject.

### TECHNICAL FIELD OF THE INVENTION

The present invention relates to new compositions and methods for the treatment of neurodegenerative disorders, and more particularly, to the characterization and therapeutic use of modulators of intracellular calcium channel signaling in cellular physiology.

### BACKGROUND OF THE INVENTION

This application claims priority to U.S. Provisional Patent Application, 60/468,160, filed May 6, 2003, the entire specification of which is incorporated herein by reference. Without limiting the scope of the invention, its background is described in connection with neurology and pharmacology, and more specifically, to drug treatments that are neuroprotective.

The list of neuroprotective agents that are either proposed or used currently for various degenerative diseases and neurotrauma are numerous and varied, both in terms of their cellular targets as well as their mechanisms of action. These treatments include, e.g., the use of cholinesterase inhibitors (Donepezil, Rivastigmine) to enhance cholinergic function in multiple forms of dementia including Alzheimer's disease (AD) (Rosier, M The efficacy of cholinesterase inhibitors in treating the behavioural symptoms of dementia. Int J Clin Pract Suppl. 2002 127:20-36); the use of non-steroidal anti-inflammatory drugs (NSAID) and the more specific, Coxib family of drugs (whose targets include the cyclooxygenases COX-1 and 2), to stave off degenerative consequences of neuroinflammation (McMurray R W and Hardy K J, Cox-2 inhibitors: today and tomorrow. Am J Med Sci. 2002 323:181-9; and McGeer P L and McGeer E G, Inflammation, autotoxicity and Alzheimer disease. Neurobiol Aging 2001 22(6):799-809).

Yet other agents enhance trophic support by increasing the expression of growth factors; the use of anti-oxidants to prevent cellular damage associated with oxidative stress (for review, see Moosmann B and Behl C; Antioxidants as treatment for neurodegenerative disorders. Expert Opin Investig Drugs. 2002 10:1407-35) and the replacement of hormones, particularly estrogen, in post-menopausal women for the prevention of such neurodegenerative diseases as Alzheimer's disease (Tang M X, Jacobs D, Stern Y, Marder K, Schofield P, Gurland B, Andrews H, Mayeux R. Effect of oestrogen during menopause on risk and age at onset of Alzheimer's disease. Lancet. 1996 348(9025):429-32). While some treatment strategies are focused toward a particular aspect of the disease, other compounds have a more diverse mode of action. For example, the compound, YM872, which has been shown in animal models to be neuroprotective against ischemic injury acts on the AMPA receptor with high specificity (Takahashi M, Kohara A, Shishikura J I, Kawasaki-Yatsugi S, Ni J W, Yatsugi S I, Sakamoto S, Okada M, Shimizu-Sasamata M, Yamaguchi T. YM872: A Selective, Potent and Highly Water-Soluble alpha-Amino-3-Hydroxy-5-Methylisoxazole-4-Propionic Acid Receptor Antagonist. CNS Drug Rev 2002 Winter;8(4):337-352).

However, despite the considerable advances at the basic science level, the translation of these numerous neuroprotective candidates to effective therapeutic interventions has been limited. For example, despite the years of using cholinesterase inhibitors for treatment of symptoms of Alzheimer's disease, it is still unclear what are the benefits of these compounds on disease progression (Windisch M, Hutter-Paier B, Schreiner E. Current drugs and future hopes in the treatment of Alzheimer's disease. J Neural Transm Suppl 2002 62:149-64). Also, the strategy to increase the expression of neurotrophins may have to be revised, given the recent finding that neurotrophins are first synthesized as pro-peptides, which have preferential affinity for the p75 receptor and may serve to promote cell death rather than survival (Lee R, Kermani P, Teng K K, Hempstead B L. Regulation of cell survival by secreted proneurotrophins. Science. 2001 294(5548): 1945-8).

Furthermore, neurotrophins are large polypeptides and thus, would be difficult to administer effectively. Estrogen replacement therapy has also issues that must be addressed, such as the risk of endometrial and/or breast cancer. Recent advances have helped circumvent some of these concerns, such as the discovery and synthesis of equally neuroprotective non-feminizing estrogens (Green P S and Simpkins J W. Estrogens and estrogen-like non-feminizing compounds. Their role in the prevention and treatment of Alzheimer's disease. Ann N Y Acad Sci. 2000;924:93-8) and selective estrogen receptor modulators (SERMs) (164-165), have helped offer alternatives which take advantage of the beneficial effects of estrogen on the brain while minimizing the adverse effects. Also, with respect to neurotrophin research, the use of neurotrophin small molecule mimetics may alleviate some issues related to delivery of the large parent molecules (Massa S M, Xie Y, Longo F M Alzheimer's therapeutics: neurotrophin small molecule mimetics. J Mol Neurosci. 2002 19(1-2):107-11).

Thus, while improvements in current strategies continue to be made, it is clear that there is an urgent requirement for the discovery and development of therapeutic strategies, that are either novel alternative or complementary, for the treatment of cell dysfunction and death associated with neurodegenerative diseases.

### SUMMARY OF THE INVENTION

The present invention relates to new compositions and methods for the treatment of neurodegenerative disorders, and more particularly, to the characterization and therapeutic use of modulators of intracellular calcium channel signaling in cellular physiology.

More particularly, the present invention includes compositions and methods that provides neuroprotection by modulating intracellular calcium concentrations when administered to a subject, the composition having an effective amount of an N-acylethanolamine (NAE). The NAE may be provided with or in a pharmaceutically acceptable carrier and/or provided in amounts of, e.g., between about 0.01 and 500 mg/kg of the subject's weight or even between about 1 and 50 mg/kg of the subject's weight. The NAEs may be, e.g., N-acylethanolamines that are 12:0, 14:0, 16:0, 18:0 and 18:2. The N-acylethanolamine may be isolated and purified after synthesis or may be from natural stores, e.g., the NAE may be plant-derived, e.g., a plant-derived extract. Depending on the condition that is targeted, the NAE may increases or decrease the intracellular calcium release from intracellular stores of neuronal cells.

The selected NAE will depend on the specific cells that are targeted and depending on the mode of delivery, e.g., intravenous or orally, may be selected to cross the blood-brain barrier. Generally, the N-acylethanolamine is dissolved in a lipophilic pharmacophor or carrier and is suitable for intravenous injection, subcutaneous, oral, intramuscular, rectal, vaginal, pulmonary, etc., administration.

The present invention includes a method for treating neurodegenerative conditions, the method including the step of administering to a subject in need thereof a composition having an effective amount of an N-acylethanolamine, e.g., in a pharmaceutically acceptable carrier, and in an amount that depends on the level of modulation of intracellular signaling and the weight of the subject if used in vivo, or when used in vitro measured by its concentration.

Depending on the extent of prevention or therapy, the composition may be carried out over a period of at least about 3, 7, 14 days or more, whether before, during or after the appearance or concern over the disease or condition that is to be treated. For example, the composition may be administered one or more times daily over a predetermined period. Examples of conditions that may be treated include a wide range of neurodegenerative conditions that results from changes in the level or extent of intracellular calcium channel signaling, e.g., ischemic cerebral trauma in a human or other mammal. In a method for treating ischemic cerebral trauma, the method includes administering to a subject in need thereof a composition with an effective amount of a plant-derived N-acylethanolamine, e.g., administered no later than about 24 hours after the occurrence of said ischemic cerebral trauma.

More generally, the present invention may be used in a method for inhibiting apoptosis under ischemic conditions in an individual in need of such inhibition by administering to the individual an effective amount to inhibit apoptosis under ischemic conditions of a composition that includes at least one N-acylethanolamine and a pharmaceutically acceptable carrier. The modulation of intracellular calcium concentration may be induced or effected by administering to a cell an effective amount of at least one N-acylethanolamine. The neuroprotection against ischemia provided by the present invention is achieved by administering to a subject an effective amount of at least one N-acylethanolamine to protect the cerebral cortex and the basal ganglia. In one specific embodiment, the ischemic injury is prevented without the activation of cannabinoid receptors.

The present invention includes a compound that provides neuroprotection having the following formula: 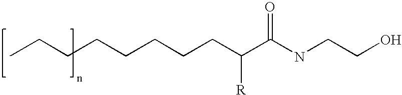
where: x is 1, 2, 3, 4, 5, 6 or more;
and R is an alkyl, an aminoethanol or an aminoalcohol; and enantiomers thereof.

Yet another compound that provides neuroprotection has the formula: 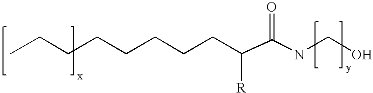
where: x is 1, 2, 3, 4, 5, 6;
where: y is 1, 2, 3, 4, 5, 6;
where R is an alkyl, an aminoethanol or an aminoalcohol; and enantiomers thereof.

Another embodiment of the present invention is a method for treating a condition in a subject, the method having the step of administering to a subject in need thereof a composition comprising an effective amount of a plant-derived N-acylethanolamine, wherein the conditions is selected from the group consisting of Alzheimer's disease, stroke, traumatic head and spinal cord injury, glaucoma, retinal ischemia, cardiac failure and ischemia and cancer. NAEs may be administered prior to, during, or after the observation of symptoms of diseases involving perturbation of the intracellular calcium homeostasis and to prevent the progression of the condition. Another methods of the present invention modulates the intracellular calcium channel of a neuronal cell in a host by determining the level of intracellular calcium channel signaling in the host and administering to the host a formulation containing an NAE, only if the level of signaling needs modulation. The level of intracellular calcium channel signaling is determined is suspected of having Alzheimer's disease, stroke, traumatic head and spinal cord injury, glaucoma, retinal ischemia, cardiac failure and ischemia and cancer and may be treated by providing in chronic or acute manner an effective amount of N-acylethanolamine, e.g., between about 1 and 50 mg/kg of the subject's weight or even between about 0.01 and 500 mg/kg of the subject's weight. The N-acylethanolamine is selected from the group consisting of N-acylethanolamine 12:0, 14:0, 16:0, 18:0 and 18:2, an alkyl at C-2, an aminoethanol or an aminoalcohol and enantiomers thereof, e.g., isolated and purified from a plant or a plant-derived extract. The N-acylethanolamine may be plant-derived and provided as a nutritional supplement.

### BRIEF DESCRIPTION OF THE DRAWINGS

For a more complete understanding of the features and advantages of the present invention, reference is now made to the detailed description of the invention along with the accompanying figures and in which:

FIGS. 1A to 1D shown the identification and analyses of NAEs in lipid extracts from seeds of several species of higher plants; FIG. 1A shows the relative abundance of individual molecular species is shown in FIG. 1A; FIG. 1B shows the structures of major NAEs identified in plant extracts; FIGS. 1C and 1D shows electron impact mass spectra (EIMS) of NAE18:2 (as TMS-ether (1C)) identified in pea seed extracts compared with the EIMS of synthetic NAE18:2 (1D), where the NAEs are denoted by the number of carbons in their acyl chain followed by the number of double bonds; and FIG. 1E shows the base structure of the NAEs of the present invention;

FIGS. 2A to 2C show representative normal-phase HPLC fractionation of lipids extracted from cottonseed meal;

FIG. 3 are representative single channel traces at various NAE: 18:2 concentrations;

FIG. 4 is a graph that shows the dependence of RyR2 activity on cytosolic NAE 16:0 concentration, measured at pCa 6. n=3 for each group;

FIG. 5 is a graph that summarizes the normalized open probability of ICCs in the presence and absence of NAE 12:0. n=3 for each group;

FIGS. 6A to 6C are primary isolated and cultured hippocampal neurons were exposed to 100 μM L-Glutamate. FIG. 6A shows the DIC image of a neuron and the fluorescence of the calcium indicator dye fluo-3 in the same cell at resting levels (6B) and after L-Glutamate stimulation (6C; scale bar: 25 μm). FIGS. 6D is a graph that shows a typical response of a neuron to L-Glutamate stimulation (arrow) under vehicle control conditions, whereas FIG. 5E is a graph that shows the response of a neuron to the same stimulus after preincubation of the cell with 100 μM NAE 16:0 for 30 min before L-Glutamate stimulus (arrow);

FIG. 7 is a Western blot showing the effects of 60 min exposure of mouse retina tissue in vitro to progesterone (A: vehicle control, B: 100 nM progesterone+15 μM LY294002, C: 100 nM progesterone) (Immunoreactivity for phosphothreonine residues on the IP3R is increased—arrow indicates IP3R band of approx. 250 kDa);

FIG. 8 is a Western blot showing the ability of NAEs to elicit the activation of signal transduction pathways relevant to the promotion of cell survival (or the prevention of cell death);

FIG. 9 shows the NAE 12:0 dose-dependently protects HT22 neurons from L-glutamate toxicity (the number of dead cells after L-glutamate insult is significantly reduced in the presence of NAE 12:0);

FIG. 10 shows the NAE 12:0 dose-dependently protects HT22 neurons from L-glutamate toxicity (the number of dead cells after L-glutamate insult is significantly reduced in the presence of NAE 12:0);

FIG. 11 is a graph that shows that NAE 18:2 dose-dependently protects HT22 neurons from L-glutamate toxicity; and

FIGS. 12A and 12B show the effects of NAE 16:0 on cerebral ischemia-reperfusion injury in ovariectomized (OVX) rats.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

All technical and scientific terms used herein have the same meaning as commonly understood by one of ordinary skill in the art to which this invention belongs, unless defined otherwise

The method of the present invention is adapted for the treatment of ischemic brain injury, such as a stroke or those injuries associated with, and secondary to, traumatic brain damage, in which “adapted for” is used to describe those compounds that are specifically selected and prepared for the method of the present invention and includes, without limitations, e.g., a compositions and method for the treatment of ill patients who must meet stringent requirements to be included as patients with ischemic brain injury. In addition, pharmaceutically effective doses of the mixture are discussed, e.g., “pharmaceutically active” is construed in the context of the treatment of ischemic cerebral damage, Alzheimer's disease, stroke, traumatic head and spinal cord injury, glaucoma, retinal ischemia, cardiac failure and ischemia and cancer, etc., and that are neuroprotective when analyzed and evaluated at the molecular level, in neuronal cell lines, and in vivo as models of neurotoxic insults and neurodegeneration and that results from the NAE affecting in a dose-dependent and even isoform-dependent regulation of intracellular calcium channels (ICC). Neuroprotection may be measured in model systems, e.g., L-glutamate mediated neurodegeneration by preventing programmed cell death.

As used herein, the term “effective amount” is used to describe the amount of active agent that modulates the release of calcium by intracellular calcium channels in neuronal or neural-derived tissue. Depending on the ICC isoforms, one or more NAEs may be administered to the patient to modify the intracellular calcium response. As used herein the term “lipophilic pharmacophor” is used to describe a plant protective agent that is used as a carrier for the NAE. The NAE may be provided in a carrier, e.g., a pharmaceutically effective carrier that aids in the delivery of the NAE.

As used herein, the term “subject” is intended to include living organisms in which certain conditions as described herein can occur. Examples include humans, monkeys, cows, sheep, goats, dogs, cats, mice, rats, and transgenic species thereof. In one embodiment, the subject is a primate, e.g., a human. Other examples of subjects include experimental animals such as mice, rats, dogs, cats, goats, sheep, pigs, and cows. The experimental animal may be an animal model for a disorder, e.g., a transgenic mouse with an Alzheimer's-type neuropathology or a normal animal or cells from an animal that have been treated with a compound or compounds that trigger a “disease-like” condition, e.g., administration of L-glutamate. A subject may be a human suffering from a neurodegenerative disease, such as Alzheimer's disease, or Parkinson's disease.

The NAEs may be administered, e.g., orally or by subcutaneous, intravenous, intraperitoneal, etc., administration (e.g. by injection). Depending on the route of administration, the active compound may be coated in a material to protect the compound from the action of acids and other natural conditions which may inactivate the compound. When administering the therapeutic compound by other than parenteral administration, it may be necessary to coat the compound with, or co-administer the compound with, a material to prevent its inactivation as is well known in the art. For example, the therapeutic compound may be administered to a subject in an appropriate carrier, for example, liposomes, or a diluent. Pharmaceutically acceptable diluents include, e.g., saline and aqueous buffer solutions. Liposomes include water-in-oil-in-water emulsions as well as conventional liposomes.

The therapeutic compound may also be administered parenterally, intraperitoneally, intraspinally, or intracerebrally. Dispersions may be prepared in glycerol, liquid polyethylene glycols, and mixtures thereof and in oils. These preparations may contain a preservative to prevent the growth of microorganisms depending on the ordinary conditions of storage and use.

Pharmaceutical compositions suitable for injectable use include, e.g., sterile aqueous solutions (where water soluble) or dispersions and sterile powders for the extemporaneous preparation of sterile injectable solutions or dispersion. In all cases, the composition must be sterile and must be fluid to the extent for delivery using, e.g., a syringe or drip-line. Generally, the compounding (pharmaceutically acceptable carrier and/or salt form (if any)) must be stable under the conditions of manufacture and storage and must be preserved against the contaminating action of, e.g., microorganisms such as bacteria and fungi. A carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, e.g., by the use of a coating such as lecithin, by the maintenance of the required particle size in the case of dispersion and by the use of surfactants. The composition may also include antibacterial and antifungal agents, for example, parabens, chlorobutanol, phenol, ascorbic acid, thimerosal and the like. In many cases, it will be preferable to include isotonic agents, for example, sugars, sodium chloride, or polyalcohols such as mannitol and sorbitol, in the composition. Prolonged absorption of the injectable compositions may be achieved by including an agent that delays absorption, for example, aluminum monostearate or gelatin.

Sterile injectable solutions for use with the present invention may be prepared by incorporating the NAEs of the present invention at an appropriate amount and in an appropriate solvent with one or a combination of ingredients described above followed by filtered sterilization. Generally, dispersions may be prepared by incorporating the therapeutic compound into a sterile carrier which contains a basic dispersion medium and the required other ingredients from those enumerated above. In the case of sterile powders for the preparation of sterile injectable solutions, e.g., by vacuum drying and freeze-drying, which yields a powder of the active ingredient (i.e., the therapeutic compound) plus any additional desired ingredient from a previously sterile-filtered solution thereof.

The NAEs may be orally administered, e.g., with an inert diluent or an assimilable edible carrier. The NAEs may also be included with other ingredients enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the NAEs in the final preparations may, of course, be varied to deliver the amount of NAE in a therapeutically useful composition such that a suitable dosage is obtained.

It is especially advantageous to formulate parenteral compositions in dosage unit form for ease of administration and uniformity of dosage. Dosage unit form as used herein refers to physically discrete units suited as unitary dosages for the subjects to be treated, i.e., each unit includes a predetermined quantity of NAE(s) calculated to produce the desired therapeutic effect in association with the required pharmaceutical carrier. The specifications for the dosage unit of the NAEs of the present invention are dictated by, and directly dependent on, e.g., the unique characteristics of the NAE(s) and the particular therapeutic effect to be achieved and (b) the limitations inherent in the art of compounding such an NAE(s) for the treatment of a selected condition in a subject.

Active compounds are administered at a “therapeutically effective dosage” are those sufficient to treat a condition associated with a “condition” in a “subject.” For example, a “therapeutically effective dosage” reduces the amount of symptoms of the condition in the infected subject by at least about 20%, more preferably by at least about 40%, even more preferably by at least about 60%, and still more preferably by at least about 80% relative to untreated subjects. For example, the efficacy of a compound can be evaluated in an animal model system that may be predictive of efficacy in treating the disease in humans, such as the model systems shown in the Examples and Figures hereinbelow.

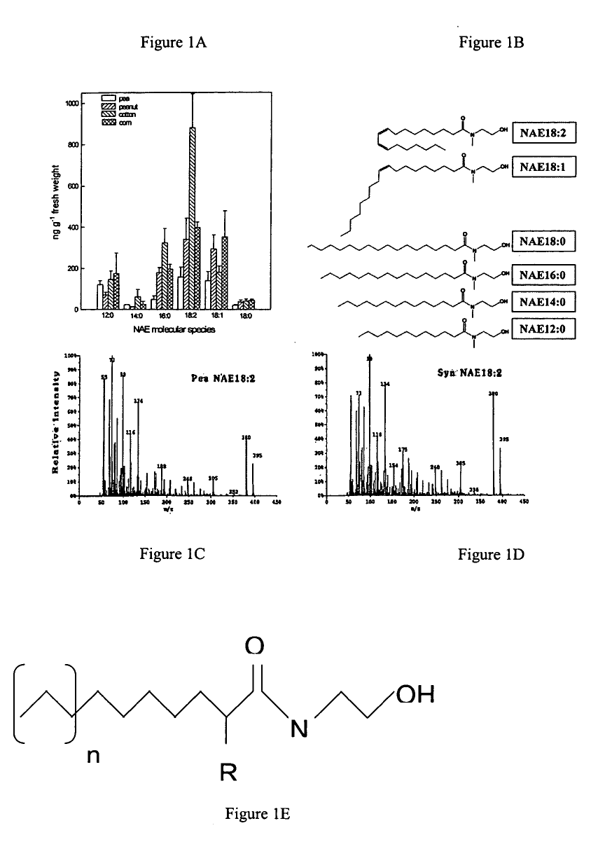

Studies were conducted to demonstrate that N-acylethanolamines, e.g., from plant tissues have neuroprotective effect and to develop and implement novel therapies for neurological disorders. The inventors have identified, characterized and used various NAE molecular species in higher plants, and has developed procedures for the routine, reproducible quantification of these lipids in relatively crude plant lipid extracts (5, 113). These studies support ongoing interests in the physiological role of NAEs in plant cells, but also form the basis for accurate quantification of these metabolites in natural products for the purposes of standardization. It is interesting that different plant tissue sources contain different NAE species, with seeds being particularly rich in NAE 18:2. FIGS. 1A to 1E show the identification, structure and analysis of NAEs in lipid extracts from seeds of several species of higher plants. Relative abundance of individual molecular species (FIG. 1A). Structures of major NAEs identified in plant extracts (FIG. 1B). Electron impact mass spectra (EIMS) of NAE18:2 (as TMS-ether) identified in pea seed extracts compared with the EIMS of synthetic NAE18:2 (FIGS. 1C and 1D). NAEs are denoted by the number of carbons in their acyl chain followed by the number of double bonds. These quantitative procedures have been extended to include novel sources of bioactive, lipid species, particularly in lower plants and algae, for which no information currently exists. FIG. 1E shows the base structure of the NAEs of the present invention, 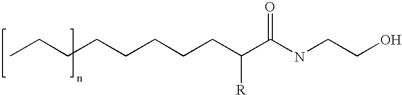
where: y is 1, 2, 3, 4, 5, 6 or more; and r is an alkyl, e.g., H, CH₃, CH₂CH₃, CH₂CH₂CH₃, CH₂CH₂CH₂CH₃, an aminoethanol or an aminoalcohol and enantiomers thereof, etc.

Yet another structure of an NAE of the present invention is: 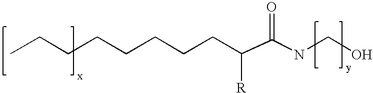
where: x is 1, 2, 3, 4, 5, 6; y is 1, 2, 3, 4, 5, 6; and R is an alkyl, e.g., H, CH₃, CH₂CH₃, CH₂CH₂CH₃, CH₂CH₂CH₂CH₃, an aminoethanol or an aminoalcohol and enantiomers thereof.

Briefly, the ICCs of the present invention include at C-2 of the parent NAE with, e.g., small alkyl (Me, Et, Propyl, Butyl) group, aminoethanols and aminoalcohols, including enantiomers thereof. For example, the aminoethanol group in NAE may be replaced with a different aminoalcohol. Such alternative head groups have been reported for anandamide analogues (Khanolkar, A. D., Abadji, V., Lin, S., Hill, A. G., Taha, G., Abouzid, K., Meng, Z., Fan, P., & Makriyannis, A. Head group analogs of arachidonylethanolamide, the endogenous cannabinoid ligand. J Med Chem, 39, 4515-19 (1996)), relevant portions incorporated herein by reference. In addition to synthetic sources of NAEs, another source are extracts from plant materials have been prepared which, depending on the species and tissue source, contained a varied composition of bioactive NAEs. Synthetic and/or modifications of NAEs from extracts may also be generated, as such, these enantiomers and preparations of R and/or S enantiomers and mixtures thereof may be used with the present invention.

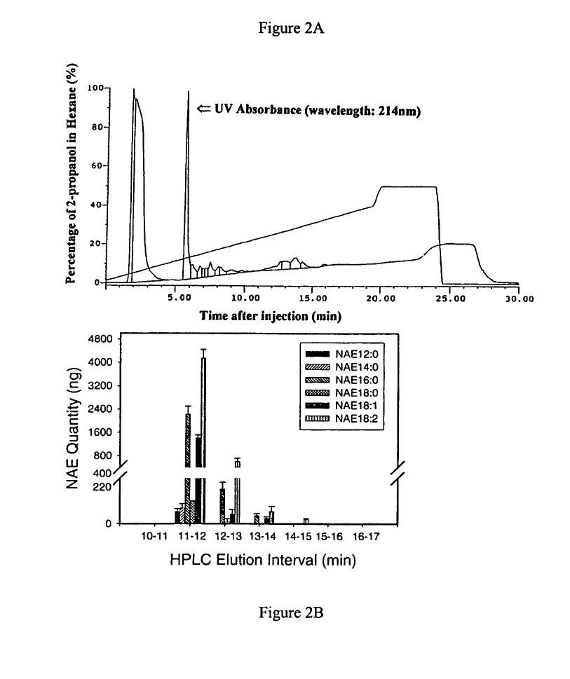

FIGS. 2A to 2D show representative normal-phase HPLC fractionation of lipids extracted from cottonseed meal. Lipids dissolved in chloroform were subjected to normal phase HPLC (4.6×250 mm Partisil 5 column, Whatman; model 712 HPLC system, Gilson) and eluted with a linear gradient of 2-propanol (0 to 40% over 20 min) in hexane (FIG. 2A). Eluting material was monitored by UV absorbance at 214 nm, and NAE types quantitatively eluted between 11 and 15 min (FIG. 2B). NAE types were identified and quantified by GC-MS.

These extracts have been analyzed by GC-MS quantification procedures. In the seed tissues examined to date, the fatty acids that are common constituents of membrane lipids in those seeds also occur as part of the NAE fraction. The HPLC fractionation scheme was based on that developed originally by Piomelli and co-workers for NAE analyses in animal tissues (114), and modified somewhat for the fractionation of plant lipids.

Electrophysiological recording of ICCs is a well established technique used to measure the effects on calcium levels based on external stimuli (83, 115-117). The planar lipid bilayer technique has been used by the principal investigator to determine the function of ICCs in neurons and non-neuronal cells. The inventors isolated type 2 IP3R from nuclei of HepG2 cells using the same techniques described herein. These studies represent the first determination of the biophysical properties of native type 2 IP3R isolated from cells. All previous studies on type 2 IP3R used artificially expressed type 2 IP3R of cells that had been transfected with the respective cDNA (118-119). Single channel activity was examined using the Planar Lipid Bilayer Membrane technique. The channels showed typical IP3R pharmacology; they were activated by IP3 and Ca2+ and were blocked by addition of heparin (50 μg/ml). The type 2 IP3R showed much higher sensitivity to IP3 than the type 1 IP3R (half maximal activation at 500 nM; 119-120): Channel activity was initiated by cytosolic IP3 concentrations of 10 nM and higher. The studies showed a sigmoidal dependence on cytosolic IP3 concentrations with half maximal activation at 64 nM (data not shown), similar to the EC50-value determined for recombinant type 2 IP3R (58 nM; 118). In other studies the effects of cytosolic Ca²⁺ concentrations on type 2 IP3R channel activity were also determined and it was found that the channel is activated like the type 1 IP3R by sub-micromolar concentrations of Ca²⁺ (119-120). However, further increases in cytosolic Ca²⁺ did not inactivate the channel. Maximal activity was reached at 10-30 μM Ca²⁺ (data not shown). The lack of feedback inhibition by high concentrations of Ca²⁺ is again similar to what has been observed in the recombinant type 2 IP3R (118, 121). Addition of excess amounts of calmodulin, the mediator of Ca2+ inhibition in type 1 IP3R (122), did not change this behavior of type 2 IP3R in our preliminary experiments indicating that the function of the inhibitory Ca²⁺ binding site in type 2 IP3R is not preserved. Both the increased sensitivity to IP3 and the lack of Ca2+-induced inactivation distinguishes the type 2 IP3R from the well-characterized type 1 IP3R. By isolating the receptor with its associated proteins from a functional cellular environment, properties influenced by accessory proteins can be readily assessed with single channel electrophysiology. Data obtained from these experiments analyzing the single channel characteristics of ICCs will provide the biophysical information necessary to correlate intracellular signaling data with function in live cells and in vivo, as shown hereinbelow.

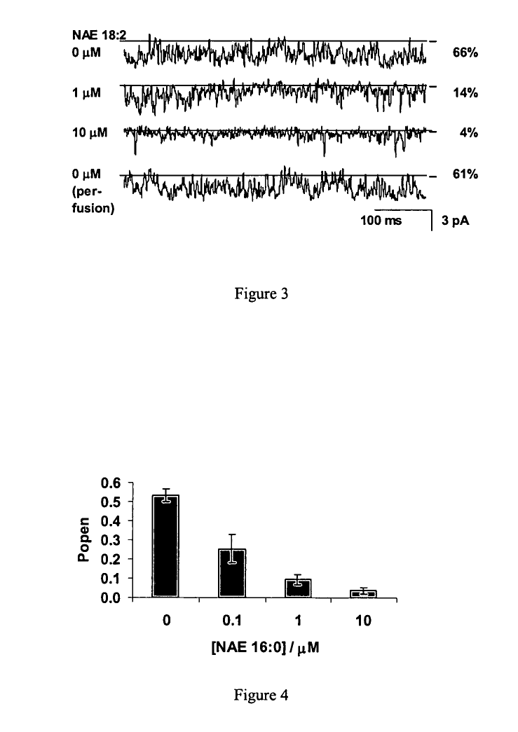

FIG. 3 are representative single channel traces at various NAE:18:2 concentrations; Dependence of RyR2 activity on cytosolic NAE 18:2 concentration. Representative single channel traces of RyR2 at various NAE 18:2 concentrations and after removal of NAE 18:2 are shown. Activity was measured at pCa 6 and bars to the right/dotted lines indicate zero current baselines. % values to the right indicate open probability.

The central discoveries of this application are that NAEs exert protective effects on neurons through the modulation of ICCs. The components and mechanisms of neuroprotection mediated by these NAEs may be analyzed and evaluated at the molecular level, in neuronal cell lines, and in vivo as models of neurotoxic insults and neurodegeneration. In particular, the effect of NAEs may be evaluated for their ability to prevent cell death and elicit signaling pathways related to neuroprotection. These studies will use a combination of immunochemistry, single channel electrophysiology, neuroprotection assays, analyses of quantitative and qualitative changes in intracellular signaling molecules and optical imaging of intracellular Ca2+ concentrations.

The present invention provides the necessary foundation for the further development, characterization, evaluation and optimization of current and novel alternative or supplemental treatments for neurodegenerative diseases and acute neurotoxic insults using NAEs by correlating data from immunolocalization, analysis of neuroprotective signaling pathways, and optical Ca²⁺ imaging studies.

The metabolism of lipids in biomembranes leads to the production of a vast array of molecules that regulate many cellular processes (1, 2, 150). In recent years, the identification of new lipid metabolites has made possible the more precise characterization of many signal transduction pathways in both animal and plant cells, e.g., the formation of the family of bioactive N-acylethanolamines from a membrane phospholipid precursor, N-acylphosphatidylethanolamine (NAPE; 3). For example, high concentrations of NAEs have been isolated and characterized identified by gas chromatography-mass spectrometry in seeds of a variety of higher plants. Work with NAPE and NAE metabolism in plants indicated that desiccated seeds were enriched in NAEs (6). These NAEs were metabolized rapidly during seed inhibition and germination by two competing pathways, one involving a 13-lipoxygenase for the formation of NAE-derived oxylipins, and one involving an amidohydrolase for the hydrolysis of NAEs (9), implying a physiological function for NAEs in regulating seed germination.

In mammalian brain, a specific NAE species, N-arachidonylethanolamine or anandamide, is an endogenous ligand for the cannabinoid receptor (4), and it is produced by phospholipase D-type activity (3). A variety of physiological effects in animals have been attributed to anandamide (and other “endocannabinoids”, such as monoacylglycerols, MAGs) including pain inhibition, anti-proliferative activity, immune modulation and regulation of embryo implantation (10). Studies of the effects of ischemic injury on membrane lipids of mammalian tissues revealed that relatively large amounts of an unusual lipid fraction accumulated dramatically in infarcted areas (11). This fraction was identified as a mixture of N-acylphosphatidylethanolamines (NAPEs) and NAEs, and these lipids are now identified as natural constituents of vertebrates, invertebrates, certain microorganisms, and higher plants (11, 12). Accumulation of NAPEs and NAEs was initially observed as a response to tissue degeneration and phospholipid degradation, and hence these lipids were presumed to play a role in membrane protection and to promote cellular survival (11, 12).

In recent years, NAEs were shown to bind and activate cannabinoid receptors and these lipids are considered components of the endocannabinoid signaling system, which mediates an array of physiological processes in animals (13, 14). In addition, NAEs appear to influence the activity of vanilloid receptors, protein kinases, ion channels, and nitric oxide synthase (13, 14), and so the tight regulation of endogenous NAE levels in animals is important to the maintenance of normal physiological functions. Indeed, genetic studies to alter either the levels or the perception of NAEs in transgenic animals emphasized the pleiotrophic effects of these lipid mediators (15, 16). Perhaps the most extensively studied physiological role for the endocannabinoid signaling pathway to date is the regulation of neurotransmission by NAEs where release of anandamide (NAE20:4) from post-synaptic neurons modulates presynaptic neurotransmitter release (17).

The pathway for the inactivation of NAE mediators has recently been identified and relies on two components. First, a specific transporter in the plasma membrane is responsible for the facilitated uptake of NAE from the extracellular side of the plasma membrane (18). Second, an active fatty acid amide hydrolase (FAAH) is responsible for the intracellular hydrolysis of NAE to FFA and ethanolamine (3, 19). While this pathway is best characterized in neurons, it is reasonable to speculate that it operates in most cell types in which the levels of extracellular NAE are transiently regulated (10). Therefore the degradation of NAEs is important in controlling their levels and thus their signaling activity. Interestingly, some NAEs are competitive inhibitiors of anandamide degradation by the FAAH (20), which led to observations that other NAE species could potentiate the activity of anandamide in vitro and in vivo (10, 21). Consequently different combinations of NAE compositions will have differential physiological effects depending upon both their inherent endocannabinoid properties as well as their influence on the metabolism of endogenous NAEs.

Classes and physiology of intracellular Ca²⁺ channels (ICCs). Since the initial discovery and characterization of intracellular Ca²⁺ channels, their importance for the function of neurons, signal transduction and information processing has been recognized (22-30). Recent studies show that intracellular Ca²⁺ channels are crucial components of diverse processes such as learning and memory formation, secretion, gene expression, metabolism, contraction, cell death, cell proliferation, neuronal excitability, neuronal differentiation, neurogenesis and apoptosis (31-38). The important neuron-in-neuron concept developed by Berridge and co-workers (31) is one prominent way to explain a number of functions of intracellular Ca²⁺ channels in neurons. To fully understand the mechanism of action of intracellular Ca²⁺ channels as part of neuronal Ca²⁺ signaling, it is necessary to analyze the molecular function of these trans-membrane proteins at the single channel and cellular level.

The inositol 1, 4, 5-trisphosphate (IP₃) receptor (IP₃R) and the ryanodine receptor (RyR) are exclusively expressed in intracellular membranes, particularly the endoplasmic reticulum (ER) membrane. These proteins each form tetrameric complexes and share substantial sequence homology in their functional domains (28). A number of molecularly and physiologically distinct isoforms and splice variants are known for both types. Despite the fact that biophysical data for specific isoforms and the localization of individual subtypes are available (29-30, 39-42), isoform specific agonists or antagonists have not been established. The number of physiological agents modulating intracellular Ca²⁺ channels present in the cytosol or the lumen of the ER is limited. Recent reviews and reports have summarized the importance of endogenous ligands of both the RyR (43-46) and the IP3R (41), such as ATP, Ca²⁺, cADPR, IP3, and lipophilic messenger substances including arachidonic acid and leukotriene B4 (44). The activity of both the RyR and the IP3R are strongly dependent on the level of cytosolic free Ca²⁺ (47-48), the presence of ATP (24, 49) and the concentration of Ca²⁺ in the ER lumen (45, 50). Recently, an additional class of ICC, polycystin-2, an ER membrane protein of the transient receptor potential channel superfamily has been isolated and characterized (77, 83).

In contrast to the RyR, which can be active in the presence of adequate amounts of cytosolic free Ca²⁺ alone, the IP3R is truly a ligand-gated Ca²⁺ channel. Both cytosolic free Ca²⁺ and IP3, generated by the activity of phospholipase C, are necessary for the activation of the IP3R (12). External messenger substances, such as neurotransmitters or hormones, activate tyrosine kinase- or G-protein-coupled receptors located in the plasma membrane (51). This signaling step stimulates the hydrolyzation of membrane-associated phosphatidyl inositol (4,5) bis-phosphate (PIP2) by phospholipase C thus producing the diffusible second messengers IP3 and 1, 2-diacyl glycerol. IP3 binds to IP3Rs, which are found on the surface of the endoplasmic reticulum (ER), the main intracellular Ca²⁺ store. Binding of the IP3R to its ligand leads to intracellular Ca²⁺ release. The receptor itself is dependent on the cytosolic Ca²⁺ concentration and is activated by sub-micromolar Ca²⁺ concentrations. For some IP3R isoforms, a deactivation by higher Ca²⁺ concentrations was observed also leading to a well-described bell-shaped dependence of the receptor on the Ca²⁺ concentration (43). The IP3R includes three functionally distinct regions: (1) the N-terminal IP3 binding domain reaching into the cytosol (52-54), (2) the membrane-spanning region that contributes to forming the tetrameric ion channel pore (28), and (3) the regulatory domain linking the two. Several regulatory sites for phosphorylation (52, 55-56), ATP binding (57), and Ca²⁺ binding (58-59) are found in the receptor. The regulatory domain also provides sites for interactions with accessory proteins, such as calmodulin (24) and the immunophilin FK506 binding protein (FKBP) (43, 45). The important function of binding proteins for the regulation of ICCs and their pharmacological relevance in disease treatment has been shown by several studies (43, 45, 60, reviewed in 72) and was the rationale for including these proteins in the preliminary and proposed experiments of the present application.

Besides the originally identified IP3R, now known as the type 1 IP3R, two additional isoforms have been characterized: the types 2 and 3 IP3R (61-63). The three IP3R isoforms are 60-70% homologous with one another (61-62) and vary in their tissue distribution (52, 61-65). Each receptor sub-type exhibits different patterns of IP3-induced Ca²⁺ release: Ca²⁺ oscillations can occur via the type 1 receptor, whereas larger, sustained signals are seen from types 2 and 3 (66-67), which correlate with their biophysical properties, namely the lack of inhibition of types 2 and 3 IP3R by higher Ca²⁺ concentrations and the different affinities for IP3 (66-67).

RyR (Ca²⁺-induced Ca²⁺ release channels) are essential components in intracellular Ca²⁺ signaling for most cell types, including neurons (31, 35, 37-38). These large tetrameric channel proteins are homologous to, but physiologically different from, IP3R. Typically RyR channels display bell-shaped activity dependence for the cytosolic Ca²⁺ concentration similar to type 1 IP3R. Lack of activation by low, and inhibition by high cytosolic Ca²⁺ concentrations tunes RyR activity to a narrow, physiologically relevant range (28, 47, 68-71). RyRs are characterized by selectivity for cations paired with a low selectivity among cations, voltage independent channel activity and physiologically relevant interactions with a number of intracellular proteins (72) and signaling substances, such as cADPR (46), arachidonic acid and its derivatives (44), sphingolipids (73-75) and ATP (49, 76).

Signal Transduction pathways linked to the promotion of cell survival: The MAPK pathway. The Ras/Raf/MAP kinase (MAPK) pathway is a signal transduction pathway that serves to propagate and amplify an extracellular signal into a biological response. This signaling pathway is initiated through the activation of a tyrosine receptor kinase by its ligand and leads to the recruitment of specific downstream effectors within a cell. Briefly, it involves the sequential activation of Ras, a small guanine nucleotide exchange protein, followed by Raf, a serine/threonine kinase, then Mitogen-activated ERK-activating Kinase (MEK), a dual specificity kinase that subsequently phosphorylates its immediate downstream target, Extracellular-signal Regulated Kinase (ERK, also called mitogen-activated protein (MAP) Kinase (for reviews see 85, 86). The consequences of activating the MAPK pathway include: proliferation, differentiation or the promotion of cell survival, although depending on the duration of the activation (rapid onset and transient versus rapid onset and sustained), the cellular context (post-mitotic neurons versus mitotically active cells), as well as the ligand that triggers this pathway, the outcome may be different (87). For example, in normally proliferating cells, such as the adrenal medulla-derived pheochromocytoma cell line (PC12), the action of epidermal growth factor (EGF) triggers a rapid, but transient activation of the MAPK cascade whose cellular consequence is to induce proliferation (88). However, administration of the neurotrophin, NGF, to this same clonal cell line results in a rapid and prolonged activation of this pathway (88), resulting in the cessation of proliferation, cell “flattening”, and the extension of neuronal-like processes, collectively referred to as neuronal differentiation (84).

Studies that evaluated the significance of MAPK activation in cell survival have both supported (20, 89), and opposed (90, 91), the importance of specific elements within the MAPK pathway. Some of the discrepancies may, in part, be attributed to the insult being used to cause cell death or the factor being administered to promote survival. For example, growth factors capable of rescuing rat sympathetic neurons from cytosine arabinoside (AraC)-induced cell death were found to necessarily elicit ERK activation. Ciliary neurotrophic factor, which in this system did not activate ERK, was consequently incapable of promoting survival following AraC treatment (92). Other paradigms that implicate the MAPK pathway in affording neuroprotection include the ability of estrogen to protect cortical neurons from glutamate toxicity (20) and the ability of N-acetyl cysteine (NAC) (89) in PC12 cells to inhibit serum withdrawal-induced cell death. Activation of the MAPK pathway has also been demonstrated to influence the catabolism of amyloid precursor protein (APP), such that the soluble fragment of APP (sAPP) is favored (93, 94), rather than the generation of β-amyloid peptide, the principle component of amyloid plaques (95, 96).

The PI-3 Kinase/Akt pathway. A separate intracellular signal-transducing pathway elicited by various growth factors involves the phosphorylation of phosphoinositides by phosphoinositide (PI)-3 kinase. These phosphoinositides can then act on multiple downstream effectors whose consequent activation can lead to a diverse range of biological functions (see 97, 98 for review). One such downstream effector, that is activated by PI-3 Kinase-induced phosphatidylinositol-3,4-bisphosphate (PI-3,4-P₂), is the PKA- and PKC-related signaling protein, Akt (also known as PKB) (99). Activation of this signaling protein is implicated in a number of cellular processes. Of particular interest is its involvement in the inhibition of apoptosis (100). For example, the PI-3 Kinase pathway has been implicated in insulin-like growth factor (IGF)-1 dependent survival of granule neurons (101) and the promotion of sensory neuron survival (91).

Furthermore, overexpression of Akt can overcome trophic factor withdrawal-induced cerebellar neuron apoptosis, while expression of a dominant negative form of Akt interferes with growth factor-induced survival of these neurons (100). Recently, it has also been proposed that an abnormality in the regulation of PI-3 kinase may contribute to the pathology in AD. Post-mortem analysis of AD brains revealed that the soluble form of PI-3 kinase was significantly reduced in the frontal cortex relative to controls (102). Since activation of the PI-3 kinase pathway promotes cell survival, this observation argues that the deficit in PI-3 kinase activity may have contributed, at least in part, to the neuronal death that occurs in AD and to neuroprotection (103-110).

Currently available neuroprotective agents and the need for novel alternative or supplemental treatments. The list of neuroprotective agents that are either proposed or used currently for various degenerative diseases and neurotrauma are numerous and varied, both in terms of their cellular targets as well as their mechanisms of action. These treatments include: the use of cholinesterase inhibitors (Donepezil, Rivastigmine) to enhance cholinergic function in multiple forms of dementia including Alzheimer's disease (AD; 151); the use of non-steroidal anti-inflammatory drugs, NSAIDs, and the more specific, Coxib family of drugs (whose targets include cyclooxygenase (COX)-1 and 2), to stave off degenerative consequences of neuroinflammation (152-153); the enhancement of trophic support by increasing the expression of growth factors; the use of anti-oxidants to prevent cellular damage associated with oxidative stress (for review, see 154) and the replacement of hormones, particularly estrogen, in post-menopausal women for the prevention of such neurodegenerative diseases as Alzheimer's disease (155). While some treatment strategies are focused toward a particular aspect of the disease, other compounds have a more diverse mode of action. For example, the novel compound, YM872, which has been shown in animal models to be neuroprotective against ischemic injury (156), acts on the AMPA receptor with high specificity.

However, despite the considerable advances at the basic science level, the translation of these numerous neuroprotective candidates to effective therapeutic interventions has been limited. For example, despite the years of using cholinesterase inhibitors for treatment of symptoms of Alzheimer's disease, it is still unclear what the benefit of these compounds on disease progression are (161). Also, the strategy to increase the expression of neurotrophins has to be revised, given the recent finding that neurotrophins are first synthesized as pro-peptides, which have preferential affinity for the p75 receptor (162), and may in turn, serve to promote cell death, rather than survival. Further, neurotrophins are large polypeptides and thus, would be difficult to administer effectively. Estrogen replacement therapy has also issues that must be addressed, such as the risk of endometrial and/or breast cancer. Recent advances have helped circumvent some of these concerns, such as the discovery and synthesis of equally neuroprotective non-feminizing estrogens (163) and selective estrogen receptor modulators (SERMs) (164-165), have helped offer alternatives which take advantage of the beneficial effects of estrogen on the brain while minimizing the adverse effects. Also, with respect to neurotrophin research, the use of neurotrophin small molecule mimetics (166) may alleviate some issues related to delivery of the large parent molecules. Thus, while improvements in current strategies continue to be made, it is clear that there is an urgent need for the discovery and development of therapeutic strategies, that are either novel alternative or complementary, for the treatment of cell dysfunction and death associated with neurodegenerative diseases. The present inventors have found, isolated and characterized a group of naturally occurring NAEs as targets for drug development and mechanistic studies (NAE 12:0, NAE 14:0, NAE 16:0, NAE 18:0 and NAE 18:2)

NAEs differentially regulate the activity of ICCs. First, type 2 RyR were isolated from mouse. Briefly, single channel activity was examined using the Planar Lipid Bilayer Membrane technique. The channels showed typical RyR pharmacology; they were activated by Ca²⁺ and were blocked by addition of ruthenium red (20 μM). Channel activity was initiated by cytosolic Ca²⁺ concentrations of 10 nM and higher. The channel showed a bell-shaped dependence on cytosolic Ca²⁺ concentrations with maximal activation at pCa 5.5. Addition of NAE 16:0 dose-dependently decreased RyR2 channel activity (FIG. 4). FIG. 4 shows the dependence of RyR2 activity on cytosolic NAE 16:0 concentration, measured at pCa 6. n=3 for each group. Both dwell time and channel open frequency were reduced significantly by addition of 0.1 μM NAE 16:0 leading to a reduction in channel open probability. Higher, micromolar concentrations of NAE 16:0 lead to an almost complete block of channel activity (FIG. 4). NAE 16:0 did not affect single channel conductance (110±8 pS) and amplitude (3.9±0.1 pA).

Next, the effect of NAE 12:0 on mouse RyRs was determined. NAE 12:0 dose-dependently and isoform-specifically regulated the function of RyRs and represent the first pharmacological tool to differentially manipulate individual intracellular calcium channel isoforms (FIG. 5). As shown in FIG. 5, the Normalized open probability of ICCs in the presence and absence of NAE 12:0. n=3 for each group. Based on these results it is possible, for the first time, to specifically target drugs that modulate intracellular calcium signaling to specific tissues or organs depending on the expression patterns of ICCs. While the dependence of RyR types I and II on the cytosolic Ca²⁺ concentration as well as the single channel conductances were unaltered in the presence of NAE 12:0, channel activity was changed significantly (FIG. 5). Measured at sub-maximally activating Ca²⁺ concentrations (RyR type I: pCa 6; RyR type II: pCa 6.5) 500 nM NAE 12:0 increased RyR type I activity by 42% whereas the same concentration decreased RyR type II activity by 64% (FIG. 5).

Similar effects were observed for another NAE species, NAE 18:2. Neither NAE 12:0 nor NAE 18:2 changed single channel conductance and amplitude of the two RyR subtypes (RyR 1 and 2) investigated. NAE 16:0 was used in many of the subsequently described preliminary experiments, because of the high potency it exhibited at the single channel level as well as due to the fact that it shows no affinity to and activity on cannabinoid receptors. Based on these results it is possible to image optically the effect of NAEs on intracellular Ca²⁺ concentrations, videomicroscopy, confocal laser scanning microscopy, and data analysis of Ca²⁺ transients in subcompartments of living cells (83, 115, 123-125). Both single wavelength (high sensitivity) and ratiometric (quantitative analysis) fluorescent Ca²⁺ indicator dyes may be used. Various parameters may be studies, such as substrate adherence, dye concentration, and culture conditions, have been optimized for measuring acutely isolated neurons as well as neuronal cell lines, as will be known to the skilled artisan. NAEs influence intracellular Ca2+ homeostasis. Next, studies were conducted using primary cultures of hippocampal neurons to determine the effects of NAEs on intracellular Ca²⁺ signaling. In these studies, the influence of a bath-based application of NAE 16:0 on intracellular Ca²⁺ signaling of primary hippocampal neurons was analyzed. Hippocampal neurons like most other CNS neurons express RyR isoform 2. Some neurons also express RyR3 and RyR1 is found predominantly in glial cells.

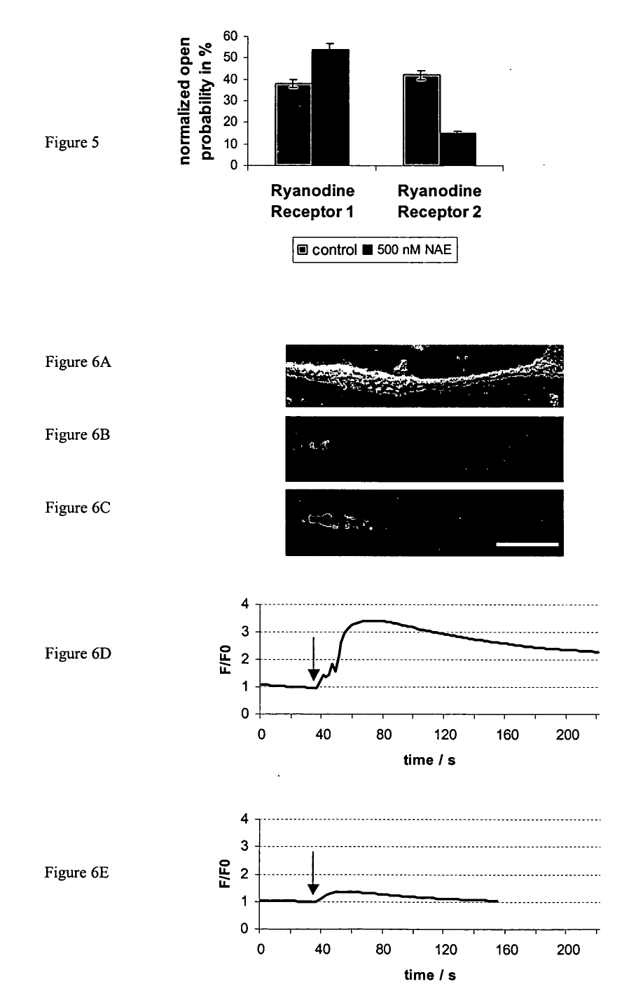

FIGS. 6A to 6E demonsatrate the effect of NAEs on primary isolated and cultured hippocampal neurons were exposed to 100 μM L-Glutamate. FIG. 6A show the DIC image of a neuron and the fluorescence of the calcium indicator dye fluo-3 in the same cell at resting levels (FIG. 6B) and after L-Glutamate stimulation (FIG. 6C; scale bar: 25 μm). FIG. 6D shows a typical response of a neuron to L-Glutamate stimulation (arrow) under vehicle control conditions, whereas FIG. 6E shows the response of a neuron to the same stimulus after preincubation of the cell with 100 μM NAE 16:0 for 30 min before L-Glutamate stimulus (arrow). It was found that NAEs influence the stimulus-induced Ca²⁺ signaling in neurons. When the hippocampal neurons were stimulated with L-glutamate, this triggers an influx of extracellular Ca²⁺ and a prolonged release of Ca²⁺ from intracellular stores (FIG. 6D). When cells were incubated with NAE 16:0 prior to stimulation with L-glutamate both amplitude and duration of Ca²⁺ transients were significantly decreased (FIG. 6E). This correlates well with the finding that NAE16:0 decreases the open probability of RyR2. These studies indicate that NAEs can influence changes in the intracellular Ca²⁺ concentration and can modulate neuronal response mechanisms to external stimuli and/or neurotoxic insults.

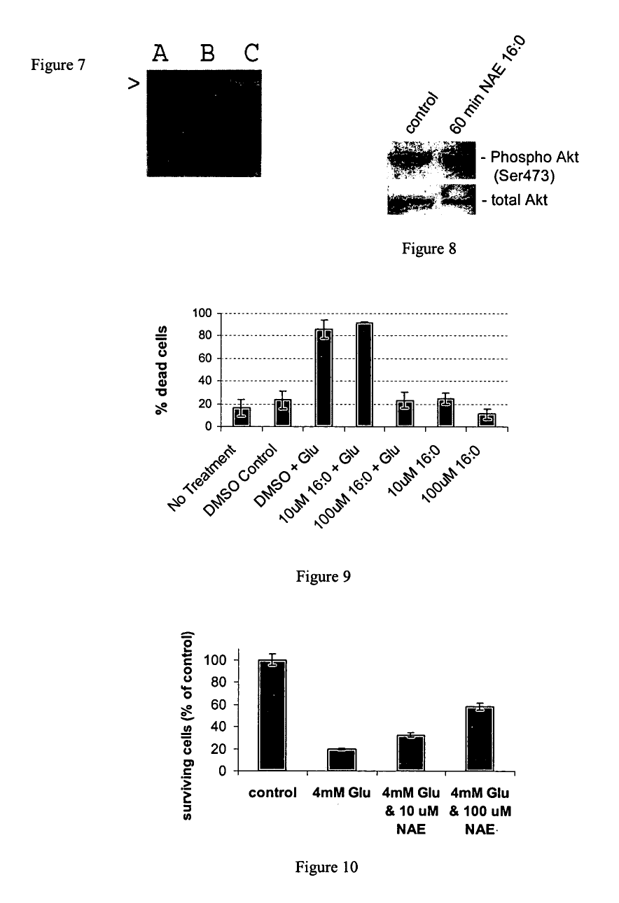

Chemo-luminescence-enhanced Western blotting as a sensitive method to evaluate changes in intracellular signaling. A crucial factor in the determination of signaling proteins mediating neuroprotection is the specificity of reagents used for immunolocalization. As disclosed herein, a number of antibodies identified and characterized (Tables 1 and 2) and the conditions that allow full identification of signaling proteins mediating NAE responses through NAEs in neurons provided. Using the sensitive chemo-luminescence-enhanced Western blotting technique, changes were detected in signaling protein composition. Furthermore, immunodetection methods allow an analysis of the changes in the phosphorylation status of signaling proteins in experiments assessing the neuroprotective functions of NAEs. The high temporal and quantitative sensitivity of the assay system may be especially useful in our in vitro and in vivo model systems of neurotoxicity which will measure small but functionally relevant initial changes associated with neuronal damage. In additional studies, the effects of lipophilic hormones on the phosphorylation status of intracellular calcium channels was determined. Progesterone treatment of the retina led to an Akt mediated phosphorylation of the inositol-1, 4, 5, trisphosphate receptor (IP3R) at threonine residues. FIG. 6 is a Western blot that shows the effects of a 60 min exposure of mouse retina tissue in vitro to progesterone (A: vehicle control, B: 100 nM progesterone+15 μM LY294002, C: 100 nM progesterone). Immunoreactivity for phosphothreonine residues on the IP3R is increased (arrow indicates IP3R band of approx. 250 kDa).

Whereas progesterone alone induced an increase of phosphothreonine immunoreactivity on the IP3R (FIG. 7, lane C) when compared to vehicle control (FIG. 7, lane A), this effect could be specifically blocked by addition of the Akt-specific kinase inhibitor LY294002 (FIG. 7, lane B). The high temporal and quantitative sensitivity of the assay system may be especially useful in our model systems which will produce functionally relevant initial changes associated with neuronal damage.

FIG. 8 is a Western blot showing the ability of NAEs to elicit the activation of signal transduction pathways relevant to the promotion of cell survival (or the prevention of cell death). Treatment of primary hippocampal neurons with 100 μM NAE 16:0 for 60 min led to a significant increase in phosphorylated/active Akt but not in total Akt (loading control) in vitro.

NAE protects neurons in vitro from oxidative stress and glutamate toxicity. Next, the neuroprotective potential effects of NAEs were determined by culturing an immortalized hippocampal neuron cell line, HT 22, a model system used extensively. These cells exhibit a pronounced sensitivity to oxidative stress with subsequent neurodegeneration and cell death after chronic exposure to millimolar concentrations of L-glutamate. The mechanism of neurotoxicity is depletion of intracellular glutathione stores by inhibition of the cysteine transport and subsequently of glutathione synthesis. Several NAE species were tested with this assay and model system of neurotoxicity and found that treatment with NAEs significantly protects cells from L-glutamate induced toxicity (FIGS. 8 and 10). FIG. 9 shows the effect of NAE 12:0 dose-dependently protects HT22 neurons from L-glutamate toxicity. The number of dead cells after L-glutamate insult is significantly reduced in the presence of NAE 12:0. FIG. 10 is a graph that shows that NAE 12:0 dose-dependently protects HT22 neurons from L-glutamate toxicity. The number of dead cells after L-glutamate insult is significantly reduced in the presence of NAE 12:0.

It was found that NAE 16:0 (FIG. 10) showed a higher potency in neuroprotection than NAE 12:0 (FIG. 9). Both NAEs alone had no effect on neuronal viability when added to HT-22 cells even at high concentrations. As indicated by the single channel data and Ca²⁺ imaging data one possible mechanism of action is the control of excessive Ca²⁺ release from intracellular stores that is a consequence of L-glutamate induced toxicity and precedes apoptosis. In long-term Ca²⁺ imaging studies a Ca²⁺ transient 8-10 h after the L-glutamate insult is typically observed.

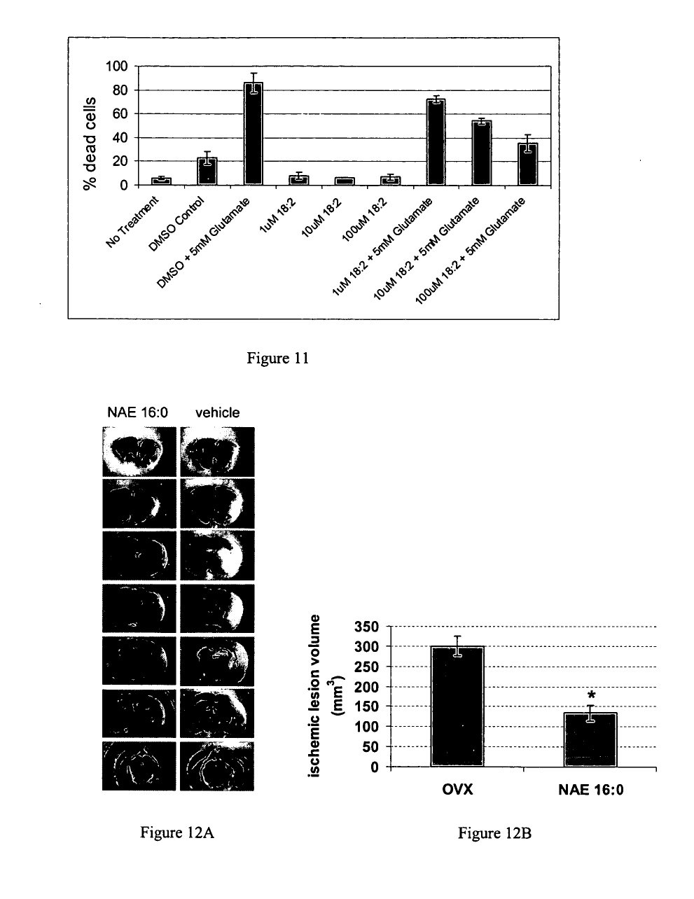

FIG. 11 is a graph that shows that NAE 18:2 dose-dependently protects HT22 neurons from L-glutamate toxicity. The number of dead cells after L-glutamate insult was significantly reduced in the presence of NAE 18:2.

NAE protects neurons in vivo from ischemic injury. Neuronal protection from ischemic injury induced by middle cerebral artery occlusion with NAEs was evaluated. Neuroprotective effects in this model system have been described for 17β-estradiol as robust (>50% protection, 126-135), seen in both male and female rats (127-135) and in mice (126) and in both transient ischemia followed by reperfusion (127-134), as well as with permanent occlusion (126, 135-136, see project 1). The observed neuroprotection by estrogens is seen when the steroid is administered by a slow-release subcutaneous implant in Silastic® pellet that produces low physiological 17β-estradiol concentrations (126, 135, 137), by a subcutaneous (127-128, 133) or an intravenous (127) injection that produces pharmacological levels of estradiol, and by an estrogen delivery system that targets estrogens to the brain (127).

For neuronal protection against ischemia, two groups of studies were preformed: (1) an acute administration schedule in which animals were injected subcutaneously 6 h prior and immediately before middle cerebral artery (MCA) occlusion; and (2) a chronic regimen with injection of NAEs 1 day prior to MCA occlusion. In these studies, NAE 16:0 was chosen because of its high potency in in vitro neuroprotection assays and in single channel electrophysiology experiments. In addition, NAE 16:0 has no affinity to and activity on cannabinoid receptors a potential mechanism of neuroprotection that can be excluded by the use of this NAE species. In both situations, NAE 16:0 effectively protected CNS tissue from MCA occlusion-induced ischemic damage. FIGS. 12A and 12B show the effects of NAE 16:0 on cerebral ischemia-reperfusion injury in ovariectomized (OVX) rats. In these studies, NAE 16:0 was dissolved in ethanol at 10 mg/kg, and administered subcutaneously (sc.) at 6 hours and again immediately before middle cerebral artery occlusion (MCAO). Cerebral ischemia-reperfusion injury was induced by 1 hr MCAO and 24 hr reperfusion. Animals were sacrificed 24 hr after reperfusion and cerebral infarct volume was determined.

FIG. 12A shows 2 mm thick coronal sections stained with 2% 2,3,5-Triphenyl-2H-tetrazolium chloride (TTC) in a 0.9% saline solution at 37° C. for 30 min followed by fixation in 10% formalin. The Pink staining indicates intact metabolically active tissue, damaged tissue appears unstained white. FIG. 12B summarizes the statistical analysis of infarct volume using Image-Pro Plus 4.1 reconstruction software. N=7/group (*: P<0.05 vs OVX). It was found that not only cerebral cortex, but also basal ganglia, were protected by NAE 16:0 unlike other small molecule neuroprotectants that primarily protect the cortex in this model system.

Analysis of the modulation of biophysical and pharmacological characteristics of ICCs by NAEs to determine if NAEs influence the functional and biophysical properties of ICCs present in neurons. Briefly, Biochemical, physiological and electrophysiological studies by others and us investigating functional processes in neurons have identified several mechanisms of action involving ICCS. Recent studies indicate that the function of ICCs strongly depends on the cellular environment and the presence and activity of proteins that interact with ICCs.

These data indicate that NAEs are potential candidate molecules for both processes using single channel electrophysiology, a powerful technique that allows the investigation of the magnitude and regulation of Ca²⁺ entry into the cytosol from intracellular stores at the molecular level. Therefore, the investigation of modulation of biophysical and pharmacological characteristics of ICCs by NAEs will identify specific functions and increase our general knowledge about the role of ICCs in neuronal signaling. Furthermore, as outlined in the Background and Significance section, data from the proposed studies may suggest potential useful treatment approaches for neurodegenerative disorders by controlling the intracellular Ca²⁺ concentration.

Patch clamp electrophysiology, the standard method for measurement of ion channel activity of ion channels on the plasma membrane, is difficult with intracellular membranes. Channels on intracellular membranes are not accessible to the patch clamp electrode without introducing significant changes to the cell. Isolated intracellular organelles are typically too small for patch clamp electrophysiology (exceptions are nuclei with their nuclear envelope membranes, especially those from Xenopus laevis oocytes; 140). Therefore, the planar lipid bilayers technique was chosen to analyze the single channel behavior of individual ICCs. This method, which in contrast to standard patch clamp electrophysiology methods, allows for greater control over the composition of the cytosolic and ER-luminal solutions and the rapid exchange of solutions on both sides of the channel protein.

In parallel to this investigation of ICCs in their native membrane and protein environment, studies using purified ICCs may be performed. Affinity-purified IP₃Rs and RyRs may be reconstituted into liposomes (142). The approach to incorporate liposomes containing purified channel proteins into planar lipid bilayers allows a determination of two additional biophysical parameters: (1) the conductivity for monovalent cations; and (2) the channel behavior in the absence of other membrane and associated proteins. Therefore, the isolation of ICCs will follow two strategies: ER membrane vesicles containing ICCs may be prepared directly from tissue or cells (type 1 IP₃R: cerebellum; type 2 IP₃R: HepG2 hepatocyte cell line, nuclear envelope membranes; type 3 IP₃R: RIN5F insuloma (beta) cell line). Vesicles may be separated from other cellular membranes and organelles by a series of differential centrifugations in the presence of protease inhibitors and under reducing conditions (77, 83). These vesicles, made up of the original ER-membrane lipids, may be used for fusion to planar lipid bilayers directly. The second set of studies will use purified ICCs that were reconstituted in liposomes of defined artificial lipid content (142). ER preparations of tissue or of cells may be solubilized using CHAPS at a non-denaturing concentration of 1%. The solubilized proteins may be purified using affinity-chromatography with resins coupled to ICC isoform specific antibodies (Pharmacia, Peapack, N.J.) and dialysis-induced reconstitution into artificial liposomes with a defined diameter of 1 μm prone to fusion with planar lipid bilayers (142). Anti-isoform specific beads may be prepared by mixing anti-host species IgG-agarose beads with the purified anti-isoform antibody (see table 1). ICC proteins may be dissociated from the antibodies and purified after elution by column gel filtration (Centri-sep, Applied Biosystems Inc., Foster City, Calif.). IP3R can be isolated in a similar fashion, but due to their specific biochemistry, a high-affinity binding capacity for heparin, immuno-affinity chromatography can be substituted with heparin affinity chromatography. All purification steps may be monitored with Western blot immunoblotting (Table 1; 83, 115, 124).

TABLE 1
Antibodies directed against ICCs used for the proposed
experiments (Chemicon International, Inc., Temecula,
CA; Sigma-Aldrich, St. Louis, MO).

| Antibodies | | | |
|---|---|---|---|
| directed against | source | Antibody type/host | dilution |
| Type 1 IP3R | Sigma-Aldrich | pAb, rabbit | 1:1000 |
| Type 2 IP3R | Chemicon | pAb, rabbit | 1:2000 |
| Type 3 IP3R | Chemicon | mAb, mouse | 1:250 |
| RyR type 1 | Sigma-Aldrich | mAb, mouse | 1:500 |
| RyR type 2 | Chemicon | pAb, rabbit | 1:2000 |
| RyR type 3 | Chemicon | pAb, rabbit | 1:1000 |

The identity of specific IP₃R isoforms may be established by the determination of biophysical characteristics of single channels and by comparison with published data. The following biophysical parameters may be tested: ion selectivity, single channel conductance, voltage-dependence, dwell times and dependence on intracellular free Ca²⁺ concentrations. By fully and specifically blocking the activity of other ICCs, specificity for one channel species may be achieved (IP₃R: no IP₃, heparin; RyR: ryanodine, ruthenium red; pc-2: cytosolic [Mg²⁺]>7 mM, lack of activating membrane potential; 77, 83, 115-117, 141). Single channel activity may be recorded, stored and analyzed using a Planar Lipid Bilayer Workstation with Axon A/D system and pClamp8 software (Warner Instrument Corporation, Hamden, Conn.; Axon Instruments, Inc., Union City, Calif.).

The effect of NAEs on ICC single channel activity may also be measured at varying cytosolic free Ca²⁺ concentrations to account for the influence of cytosolic free Ca²⁺ concentrations on channel activity. NAEs (NAE12:0, NAE14:0, NAE16:0, NAE18:0, NAE18:2, and novel non-natural NAEs that have either a C-2 alkylation or substitution of aminoethanol with different aminoalcohols; vehicle control) may be tested below, at, and above their respective physiological concentrations in plants, over a concentration range of 10 nM to 1 mM, concentrations that also have been identified as physiologically relevant for mammalian analogues. In control studies, lauric acid (Tocris Cookson Inc., Ellisville, Mo.) was tested a precursor that has been shown inactive in plant physiological applications, as well as anandamide (Tocris Cookson Inc.), the major mammalian analogue. The reason for testing a wide range of concentrations is to be able to evaluate the effects of very low physiological concentrations that are typical for intracellular messenger substances in intact cells. In addition, a range of higher concentrations may be tested to address conditions that are typically seen under pathological disease conditions, when messenger substances accumulate or their levels are high due to constant pathway stimulation.

These data may be interpreted independently using quantification of ICC single channel biophysical parameters. Application of identical acquisition parameters will provide the quantitative basis for the statistical analysis. Statistical analyses may be conducted using, e.g., standard one way or multiple ANOVA for comparisons of parametric populations using, e.g., the Statview program, which may also used for linear regression analyses of the proposed correlational hypothesis involving data sets from different experimental approaches. Changes in the ICC single channel biophysical parameters may be correlated with the activation of intracellular signaling pathways and evaluation of neuroprotection; these results may be interpreted both mechanistically and functionally.

Since the protocols for the isolation and electrophysiological measurement of ICCs have been tested for and applied to central nervous system (CNS) tissues and cell culture models in the PIs laboratory it is not anticipates that technical difficulties in determining these biophysical data. To prevent potential problems that could arise from differences in ICC subunit composition and changes in the number and type of associated proteins of ICCs from different sources it may be necessary to monitor such changes in the protein composition of ICC complexes that could potentially mimic changes at the molecular level (e.g. a lack of a subunit could produce similar changes in the open probability as a direct pharmacological modulation) by Western blot analysis (table 1). As an alternative strategy, it is possible is possible to identify molecular differences by direct pharmacological modification of ICCs and relate these results to established pharmacological protocols (reviewed in 141). Should alternative explanations for channel behavior occur at the single channel level that cannot be addressed with the pharmacological tools available the mechanisms of action may be evaluated in live cells. Further evaluation of compounds may be prioritized based on potency and ICC isoforms' relevance for CNS applications. In parallel, development of novel compounds may be guided by results from the evaluation of parent compounds.

Identifying and measuring the contribution of NAE mediated responses to intracellular Ca²⁺ signaling of neurons. It is also possible that NAEs modulate Ca²⁺ signals mediated by individual classes of ICCs in neuronal cell lines (differentiated PC12 and HT22) and in primary neuronal cultures of the hippocampus.

In order to investigate activity of signaling proteins mediating NAE responses in their native environment and to test interactions with other cellular signaling components, electrophysiological studies and signaling assays in vitro and in vivo may be combined with the analysis of NAE responses influencing the intracellular Ca²⁺ concentration using optical recordings of intracellular Ca²⁺ concentrations/Ca²⁺ imaging. The combination of these approaches, as used previously (115, 123-124), may be used to verify results from the molecular level at the cellular and animal model level. Furthermore, it may be used to evaluate the physiological relevance of biophysical data at the cellular level. L-glutamate induced neurotoxicity, and ischemia induced apoptosis of neurons are both coupled to increases in the intracellular Ca²⁺ concentration, predominantly caused by stimulation of ionotropic glutamate receptors but also by pathologically affecting the activity of mitochondria (143-145). Intracellular Ca²⁺ signaling is not the only possible signaling mechanism relevant for neurodegeneration but has been implicated as a crucial factor in a variety of neurotoxic pathways.

Using techniques and the materials known currently and methods disclosed herein it is possible to explore the qualitative and quantitative role of NAE mediated Ca²⁺ signaling in neurons while integrating the electrophysiological data and related published data. Therefore, it is possible to evaluate possible mechanisms of action of signaling proteins mediating NAE responses in a cellular environment. The signaling mechanism and processes used by neurons may be dissected out of a number of possible pathways that can be suggested from the combination of localization and biochemical data. In addition to increasing the knowledge of the basic underlying signaling processes in neurons the techniques taught herein may be used to evaluate pharmacological treatments for diseases and conditions affecting the Ca²⁺ homeostasis of neurons without undue experimentation as will be known to the skilled artisan.

As with the previous example, imaging of intracellular Ca²⁺ concentrations at the subcellular level may be performed as described previously (115, 123, 125). Briefly, neurons may be plated on coverslips and cultured for Ca²⁺ imaging as described previously (see specific methods below). Two neuronal cell lines, differentiated PC12 (124) and HT22 (146) and primary neuronal cultures of the hippocampus may be used. These models will cover two aspects involved in neurodegeneration: glutamate and calcium neurotoxicity (differentiated PC12 cells, hippocampal cultures), and oxidative stress (HT22 cells). At the same time use of these model systems allows a correlation and comparison of data, e.g., the expression of signaling proteins mediating NAE responses by neurons. Viability of cultures may be assessed with a Viability/Cytotoxicity kit for animal cells (Molecular Probes, Eugene Oreg.). This kit specifically stains intact live cells and dead cells with damaged cellular membranes using cell-permeant und cell-impermeant fluorescent dyes. Non-viable cells are recognized easily with bright field differential interference contrast microscopy as necrotic and will not be included in the experiments. Neurons may be identified in hippocampal cultures based on their morphology, antigen content and on their physiological responses to neurotransmitter receptor agonists. Cells may be exposed to L-glutamate at 5-10 mM (HT22 cells, hippocampal cultures) or 10-50 μM (differentiated PC12 cells) to induce Ca²⁺ transients and subsequent neurotoxicity.

The effect of NAEs (NAE12:0, NAE14:0, NAE16:0, NAE18:0, NAE18:2, and novel non-natural NAEs that have either a C-2 alkylation or substitution of aminoethanol with different aminoalcohols) on L-glutamate induced Ca²⁺ transients may be tested below, at and above their respective physiological concentrations, over a concentration range of 10 nM to 10 mM, concentrations that also have been identified as physiologically relevant for mammalian analogues. Higher concentrations of NAEs may be used to accommodate an expected partitioning of NAEs into the plasma membrane based on a comparison with compounds that have a similar lipophilicity. It was found that NAEs are active in the assays at physiological concentrations observed in plants or at concentrations physiologically relevant for mammalian analogues. Control studies may be conducted with lauric acid (Tocris Cookson Inc.), a precursor that has been shown inactive in plant physiological applications, as well as anandamide (Tocris Cookson Inc.), the major mammalian analogue. One reason for testing a wide range of concentrations is to be able to evaluate the effects of very low physiological concentrations that are typical for intracellular messenger substances in intact cells. In addition, a range of higher concentrations may be tested to address conditions that are typically seen under pathological disease conditions, when messenger substances accumulate or their levels are high due to constant pathway stimulation.

To test different signaling mechanisms, mitochondria-mediated oxidative stress and excitotoxicity, as well as to assess potentially clinically relevant dosing regimens cells may be pre-incubated with NAEs, NAEs may be co-applied with L-glutamate, or NAEs may be administered after the neurotoxic insult. Based on previous experiments, addition of NAEs may be at 0.5 and 2 hours before (all models), or at 0.5, 1 (all models) and 4 (HT-22 cells) hours after L-glutamate addition. It was found that cannabinoid receptors were not detected, potential alternative sites of action of anandamide analogues, on the two cell lines used as model systems. To exclude the contribution of cannabinoid receptors in hippocampal cultures, the specific cannabinoid receptor antagonists AM-251 and AM-630 (BIOMOL Research Laboratories, Inc., Plymouth Meeting, Pa.) may be used.

Regardless, the data may be interpreted independently using quantification of signaling patterns (amplitude, duration, period until half-maximal intensity is reached, slope of rise) by the automated intensity analysis software of this imaging system. Statistical analyses involves standard one way or multiple ANOVA for comparisons of parametric populations using the Statview program, which is also used for linear regression analyses of the proposed correlational hypothesis involving data sets from different experimental approaches. Changes in the signaling patterns of intracellular Ca²⁺ transients involved in steroid hormone signaling in RGCs may be correlated to interpret results both mechanistically and functionally. Data may be interpreted independently and in conjunction with results obtained hereinabove. Changes in the Ca²⁺ homeostasis neurons and NAE induced changes in signaling patterns (amplitude, duration, period until half-maximal intensity is reached, slope of rise) may be evaluated as taught herein and as will be known by the skilled artisan.

The protocols for the measurement of changes in the intracellular Ca²⁺ concentration have been tested for and applied to primary neuronal cultures of the hippocampus (FIG. 5) and cell culture models of the current proposal (124). Potential problems might also arise from the use of membrane-permeable fluorescent Ca²⁺ indicator dyes, however, other acetoxymethyl ester derivatives of fluorescent Ca²⁺ indicator dyes (fluo-4/fura-2 acetoxymethyl ester; Molecular Probes, Eugene, Oreg.) may be used and/or the loading parameters adjusted (time, concentration, co-application of pluronic acid) to determine optimal loading conditions for alternative fluorescent Ca²⁺ indicators (indo-1, Calcium Green-1&2, Calcium Orange, Calcium Crimson).

Next, the neuroprotective effects of NAEs in neuronal cell lines as models of neurotoxic insults and neurodegeneration may be determined to ascertain if NAEs mediate neuroprotective effects in neuronal cultures. As shown hereinabove, cells may be exposed to L-glutamate at 5-10 mM (HT22 cells) or 10-50 μM (differentiated PC12 cells) to induce Ca²⁺ transients and subsequent neurotoxicity. The effect of NAEs (NAE12:0, NAE14:0, NAE16:0, NAE18:0, NAE18:2, and novel non-natural NAEs that have either a C-2 alkylation or substitution of aminoethanol with different aminoalcohols) on L-glutamate induced neurotoxicity may be tested below, at, and above their respective physiological concentrations in plants, over a concentration range of 10 nM to 10 mM, concentrations that also have been identified as physiologically relevant for mammalian analogues.

As discussed above, different signaling mechanisms may be studies using mitochondria-mediated oxidative stress and excitotoxicity, as well as to assess potentially clinically relevant dosing regimens cells may be pre-incubated with NAEs, NAEs may be co-applied with L-glutamate, or NAEs may be administered after the neurotoxic insult. Based on previous experiments, addition of NAEs may be at 0.5 and 2 hours before (all models), or at 0.5, 1 (all models), 2 and 4 (HT-22 cells) hours after L-glutamate addition. The following assays may be used to assess neuronal viability and different stages of cell death: Optical assessment of changes in cell morphology, optical assessment of cell viability using fluorescently labeled dyes, TUNEL) cytochemistry, Annexin V membrane staining, LDH release assay and PARP cleavage assay (for each method see general methods, below). As above Data may be interpreted independently using quantification of assay output data. Statistical analyses will involve standard one way or multiple ANOVA for comparisons of parametric populations using the Statview program, which is also used for linear regression analyses of the proposed correlational hypothesis involving data sets from different approaches.

The protocols for the measurement and quantitation of neurotoxicity and neuroprotection have been tested for and applied to neuronal cultures of the hippocampus and cell culture models of the current proposal. Therefore, technical difficulties are anticipated in determining these data. As always, potential problems might arise from variability of outcomes based on culture conditions, however, methodical eliminatation of variable parameters may be used to minimize variability including controlling cell density, passage numbers, exposure times to drugs and media changes. One way to minimize variability is to standardize all culture parameters to established and published protocols. The amyloid beta protein toxicity and trophic factor deprivation models used extensively in project 2 may be used as alternative and supplemental models for high priority compounds to further characterize the function and molecular mechanisms of NAEs.

Next the identity of the neuroprotective signal transduction pathways elicited by NAEs in primary neuronal cultures of the hippocampus as models of neurotoxic insults and neurodegeneration may be determined. Briefly, NAEs may activate the MAPK and the PI-3 kinase pathways mediating neuroprotection of primary neuronal cultures of the hippocampus from L-glutamate induced neurotoxicity. To address this question, an in vitro model of neurotoxicity may be employed, using primary neuronal cultures of the hippocampus. Cultures may be treated with L-glutamate to induce neurotoxicity. L-glutamate, its analogs and L-glutamate specific ligands have been used in various culture systems to induce cell death and mimic consequences of ischemia. The use of this paradigm offers a relevant in vitro model for the assessment of NAE mediated neuroprotection and its signaling pathways. The two major signal transduction pathways relevant to the promotion of cell survival (or the prevention of cell death) that may be investigated in this aim have been found to be involved in neuroprotection mediated by other bioactive lipophilic molecules.

Briefly, in primary isolated cultured neurons of the hippocampus, the effect of NAEs (NAE12:0, NAE14:0, NAE16:0, NAE18:0, NAE18:2, and novel non-natural NAEs that have either a C-2 alkylation or substitution of aminoethanol with different aminoalcohols), over a range of concentrations between 10 nM and 10 mM or vehicle control, may be evaluated on their ability to elicit the phosphorylation and activation of key effectors of the MAPK and PI-3K signaling pathways. Higher concentrations of NAEs may be used to accommodate an expected partitioning of NAEs into the plasma membrane based on previous experiments with compounds that have a similar lipophilicity. The reason for testing a wide range of concentrations is to evaluate the effects of very low physiological concentrations that are typical for intracellular messenger substances in intact cells. In addition, a range of higher concentrations may be tested to address conditions that are typically seen under pathological disease conditions, when messenger substances accumulate or their levels are high due to constant pathway stimulation. To exclude the contribution of cannabinoid receptors in hippocampal cultures, the specific cannabinoid receptor antagonists AM-251 and AM-630 (BIOMOL Research Laboratories, Inc., Plymouth Meeting, Pa.) may be used in experiments and controls.

Immunoblot analysis of the Bcl-2 family of proteins will also be evaluated. Specifically, evaluating the ratio of Bcl-2 to Bax expression in the hippocampal cultures may be determined in parallel. Relative increases in the ratio of Bcl-2:Bax will reflect a neuroprotective change, whereas the converse will reflect dying cells. Apoptotic mechanisms are likely to be involved, therefore studies may be focused on proteins that are involved in apoptotic signaling and whose expression levels are functionally correlated with apoptosis. Use of the specific blockers PD98059 and LY294002, will further assess the involvement of the MAPK and PI-3K pathways in NAE-induced neuroprotective effects. For example, the ability of NAEs to elicit the activation of two signal transduction pathways relevant to the promotion of cell survival (or the prevention of cell death) may be characterized. These are the Ras/Raf/MAPK pathway and the PI-3K/Akt pathway: The Ras/Raf/MAPK pathway consists of the sequential activation of Ras, followed by Raf, then MEK and finally the activation of ERK (for review, see 85). Activated ERK can then regulate additional signaling proteins (such as Rsk) or itself translocate into the nucleus to regulate gene transcription (85). The relevance of this pathway on neuronal cell survival is supported by the work of Desire et al. (147) where in a serum-deprivation model of cell death, FGF2 treatment promoted the survival of these neurons in a bcl-x(L) and ERK-dependent manner. The activation of the PI-3 Kinase pathway has also been associated with the inhibition of apoptosis. The phosphorylation and activation of Akt, a downstream signaling element and key effector of the PI-3 Kinase pathway, has been deemed necessary and sufficient for promoting neuronal survival in the face of such insults as growth factor withdrawal. The objective is to evaluate the extent to which NAEs recruit members of the MAPK and PI-3K/Akt pathways. Detailed characterization of the ability of NAEs to activate these signal transduction pathways will serve not only to provide a novel mechanism by which these bioactive lipids elicit their rapid effects in neurons, but could also provide the basis for their neuroprotective potential.

The following studies will address and characterize NAEs' ability to activate these signal transduction pathways in hippocampal neurons. Time-course and dose-response evaluation for the effect of NAEs on ERK phosphorylation. On the 14^{th} day in vitro, the primary hippocampal neuron cultures may be treated with a specific NAE concentration (see above) for a duration of 5, 15, 30 min, 1 hr, 2 hr and 4 hr. These treatments may be compared to a 30 min treatment with BDNF (100 ng/ml), serving as the positive control, and a sham-treated control culture, serving as an indication of basal ERK phosphorylation. After the specified duration and dose of treatment, the cultures may be harvested and subject to Western blot analysis.

The effect of NAEs on the activities of upstream signaling proteins within the MAPK pathway. It has previously been demonstrated that neuroprotective drugs may not necessarily activate the same complement of signaling isoforms as those elicited by putative activators of this pathway (like the neurotrophins). The relevance of this observation is that the cellular response to NAEs may differ depending on which Raf isoform is activated, or the duration for which a particular Raf isoform is activated (148). For example, NGF, which elicits differentiation of PC12 cells and promotes cell survival following serum withdrawal, causes the prolonged activation of B-Raf, but not c-Raf (148). Thus, any isoform selectivity observed in NAEs' actions will not enhance the characterization of this novel signal transduction pathway. In view of these possible differences in isoform specific activation of this pathway, the effect of NAEs on B-Raf, c-Raf, MEK1, MEK2 and ERK may be evaluated.

In evaluating the dependence of MEK in the NAE-mediated phosphorylation of ERK, cultures will first be pre-treated for 30 min with the MEK inhibitor, PD98059 (100 μM) prior to the administration of NAEs. Should MEK be determined as necessary for the NAE-induced phosphorylation of ERK, kinase assays for the separate evaluation of the kinase activities of MEK1 and MEK2 may be performed. The concentrations of NAEs that elicited maximal phosphorylation of ERK (from the results of Experiment 1) may be used in the evaluation of kinase activities. The time points to be used for the evaluation of NAE-induced kinase activities may be abbreviated (only 3 time points) and will coincide with: the time point just before maximal NAE-induced ERK phosphorylation, the time point at maximal ERK induction, and the time point immediately after peak induction, respectively. Specific methodology for the in vitro kinase assays is provided in the General Methods section.

Evaluation of the cellular and subcellular (nuclear vs. cytosolic) distribution of NAE-induced phosphorylated ERK. Due to the heterogeneous nature of the hippocampus, which includes various cell types the cellular distribution of the phosphorylated form of ERK (phosphoERK) will also be determined in the NAE-treated cultures using immunohistochemistry together with fluorescence microscopy in order to determine the specific effects on hipppocampal neurons. NAEs, at concentrations and durations of treatment that resulted in maximal ERK or Akt phosphorylation (as determined from objective A) may be used for the immunohistochemical analysis. Three treatment groups may be evaluated: control (sham-treated), NAE-treated, and 100 ng/ml BDNF-treated for 30 min (positive control). The use of a pre-immune IgG as the primary antibody will serve as a negative methodological control, to ensure the specificity of the signal. Specific details of this immunohistofluorescence protocol are described herein. Using this method, cellular distribution as well as subcellular localization (nuclear vs. cytosolic) may be evaluated in control, NAE- and BDNF-treated cultures.

Time-course and dose-response evaluation for the effect of NAEs on Akt phosphorylation. Two major phosphorylation sites within Akt have been described (Thr308 and Ser473), and are required for the activation of Akt. Using phosphospecific antibodies to Thr308-phosphorylated Akt, and Ser473-phosphorylated Akt (table 2) independently, the ability of NAEs to phosphorylate Akt in a time- and dose-dependent manner may be evaluated. The same concentrations and time-points as those used to study the phosphorylation of ERK may be tested, and compared with the neurotrophin (BDNF) control.

It has been documented that activation of Akt may occur independently of PI-3 Kinase. For example, MAPKAP kinase 2, which is activated by the stress-associated MAPK, p38, can phosphorylate Akt on Ser473 to partially activate Akt in vitro, and that this occurs independently of PI-3 Kinase activation (85). To determine if PI-3 kinase activity is a requisite for NAEs actions on Akt, the effect of these lipids (at concentrations and durations of treatment that elicit maximal Akt phosphorylation) may be evaluated in the presence of 15 μM LY294002 (Calbiochem, San Diego, Calif.), a specific PI-3 kinase inhibitor, and compared to the effect of NAEs alone. The concentration selected for LY294002 represents a 10-fold higher concentration than the reported Kᵢ for this compound. Cultures may be pre-treated with the PI-3K inhibitor, LY294002 (15 μM) for 30 min prior to the administration of NAEs (at optimal concentrations) in the continued presence of the inhibitor. These cultures will then be harvested and evaluated for Akt phosphorylation using Western blot analysis.

Evaluation of whether inhibition of the MAPK and/or PI-3K pathways prevents NAEs protective effects against glutamate insult. Cultures pretreated (30 min) with either the MEK inhibitor, PD98059, or the PI-3K inhibitor, LY294002, or vehicle control (0.1% DMSO) may be treated with L-glutamate for 12, 18, 24, 48 and 72 hours in the continued presence of the respective NAE (or vehicle control). These time points for glutamate exposure were chosen based on studies that yielded an easily quantifiable and reproducible neurotoxic insult to hippocampal neurons that has sufficient flexibility in the extent of affected cells, based on the duration of exposure to the L-glutamate insult. Following the specified duration of treatment, the cultures may be harvested into protease- and phosphatase-inhibitor containing lysis buffer and processed for PARP cleavage. The media will also be harvested for the parallel evaluation of LDH release. Four group comparisons may be made: Between control cultures (non-glutamate treated, non kinase inhibitor pre-treated), cultures treated with glutamate alone (kill control), glutamate-treated cultures treated with NAE alone, and glutamate-treated cultures in the presence of the appropriate kinase inhibitor.

The proposed studies may be used to characterize in detail the extent to which signaling proteins within the MAPK and PI-3 Kinase pathways are used for NAEs' actions in hippocampal neurons. The resulting data may describe only a partial overlap with these growth factor signaling pathways, thereby revealing and characterizing differences between NAE-induced and growth factor-induced pathways. It is anticipated that NAEs will activate members of both neuroprotection-related signaling pathways. The results will ultimately provide new and important information regarding the mechanism by which these lipids act in the CNS. Results that document the ability of NAEs to activate these neuroprotection-related signaling pathways will undoubtedly complement the in vivo neuroprotective effects of these compounds in the ischemia model, offering possible mechanisms for their actions. The ability of NAEs to independently activate these signal transduction pathways would set the stage for the analysis of potential interactions between these compounds and other neuroprotective substances.

Immunohistofluorescence may be used to evaluate the precise cellular distribution of cells responsive to NAEs, at least with respect to ERK and Akt. Furthermore, NAEs' ability to elicit nuclear translocation of the phosphorylated ERKs will also be determined using immunohistofluorescence and will provide further insight into the biological significance of their actions. Transient activation of ERK does not result in nuclear translocation, while sustained activation of ERK, as induced by the neurotrophin, NGF (a factor that leads to differentiation and promotes the survival of PC12 cells), does lead to nuclear translocation of ERK.

As an alternative strategy, cultures may be treated with NaCN/2-Deoxy-D-glucose (2-DG) to simulate ischemia. These compounds have been used in various culture systems to mimic consequences of ischemia. Specifically, NaCN inhibits oxidative metabolism, while 2-DG serves to inhibit glycolysis and provide a rapid loss of ATP. Since NaCN also results in the activation of NMDA receptors, the use of this paradigm may offer a relevant in vitro model for ischemia-induced neuronal damage. Cultures may be treated with NAEs as described above prior to or after the application of the NaCN/2-DG treatment. Subsequently, the cultures may be treated with 1 mM NaCN/2 mM 2-DG for 2 h [modified from the method of Wilson et al. (149)] in the continued presence of the respective NAE (or vehicle control). Following this 2 h incubation, the NaCN/2-DG containing media may be switched back to serum-containing media supplemented with or vehicle (steroid-free control). Indices of cell death may be evaluated at 24, 48 and 72 hrs following initial exposure to NaCN/2-DG.

Apoptotic signaling may also be evaluated and the expression levels of proteins and second messengers may be evaluated to determine the functional correlation with apoptosis. Two likely candidates are Bcl-2 and Bax, however, should no change in these proteins be observed, the investigation may be expanded to include other members of the Bcl-2 family (i.e., Bcl-x_{L} and BAD). This alternative strategy allow the analysis the function of proteins that are involved in the majority of apoptotic signaling events. Other techniques evaluating the expression and cellular localization of hyperphosphorylated tau also provide an alternative and supplemental models for high priority compounds to further characterize the function and molecular mechanisms of NAEs.

NAEs may also be evaluated for their effect on an ischemic damage in an animal model of stroke. In addition to the results provided herein, the dose- and time-dependence of neuroprotection of cortical neurons by NAEs may be identified with biochemical, immunochemical and histological methods. Using a model for cerebral ischemia, middle cerebral artery (MCA) occlusion, the effects of NAEs on the resulting extent of neuronal damage may be further characterized. At various times after ischemia, cell viability as the outcome measure may be assessed. It has been found herein that NAEs protect neurons from neurotoxic damage. However, a systematic assessment of the dose- and time-dependence of this possible CNS protection may be determined.

Each of the following studies may use adult female Sprague-Dawley rats. Animals will either receive sham surgery (Intact group) or be ovariectomized (OVX) two weeks prior to MCA occlusion surgery. Ovariectomy may be performed to exclude neuroprotective effects of endogenous steroid hormones. The OVX rats may be divided into placebo treatment and NAE treatment using the concentrations. Silastic dosing levels that produce different physiological and pharmacological concentrations may be achieved by dissolving various concentrations of NAEs in oil. All animals will receive their treatment by Silastic tube, implanted at the time of ovariectomy and two week prior to MCA occlusion surgery. Ischemia may be induced by transient occlusion of the MCA as described previously (127-128, 132, 136-138). Following a one-hour MCA occlusion, animals may be reperfused until the time of sampling at 12, 24 and 48 hours and at 1 week. Blood samples may be obtained at the time of MCA occlusion as well as at sacrifice to determine plasma concentrations of NAEs. Next, a one-hour MCA occlusion as the optimal parameter to induce a significant, but anatomically well defined cerebral ischemia. In previous studies it was found that the duration of MCA occlusion is directly correlated to the number of apoptotic neurons. Thus, an animal model has enough flexibility to accommodate a large number of cells for an aptoptotic response.

Brains may be removed and/or fixed and neuronal damage may be assessed as described hereinabove. For example, to determine the duration of exposure to NAEs needed to protect neurons from mild ischemic damage, we will repeat the aforementioned study, but treat with NAEs at various times before the onset of MCA occlusion by s.c. injection. Animals may be ovariectomized and be treated with placebo or NAEs, for 1 week, 4 days, 1 day or 1 hour prior to the onset of MCA occlusion. Animals may be sampled at different time points to detect and quantify neuronal damage.

Based upon the preliminary data disclosed herein, which indicate that NAEs are neuroprotective, optimization of NAE treatment is expected to decrease the damage related to cerebral ischemia. Studies to optimize the action of the NAEs may be designed to maximize the effect of the NAEs based on, e.g., the timing of doses after the MCA occlusion. For example, sampling of tissue for evidence of damage at 12, 24 and 48 hours and 1 week after MCA occlusion. The timecourse may be expanded or contracted based upon the observation that NAE preventable neurodegeneration was detected in vitro at 18 hours after the neurotoxic insult. Thus, the proposed sampling times bracket this one preliminary observation point. Also, the proposed study permits a more complete determination of the short and long term effects of NAEs on neurodegeneration observed with ischemia, e.g., whether it is acute, transient or chronic (with recovery expected by the 1 week sampling time point) or permanent. The fimbria-fornix lesion/axotomy models may be used as an alternative and supplemental model for high priority compounds to further characterize the function and molecular mechanisms of NAEs.

A course of in vitro and in vivo studies for toxicity, potency and maximization of the effects of NAEs may be performed using one or more of the following techniques. Briefly, ICC proteins may be isolated from 12 individual animals for each compound (36 compounds, 12 animals each=432 animals). Estimates for animal requirements are based on the amount of tissue required to generate sufficient protein quantities for each of the ICCs to be investigated. Neurons may be isolated from 10 individual animals for each compound (36 compounds, 10 animals each=360 individual animals=60 litters or time-pregnant animals; experiments performed in triplicate=1080 animals=180 litters or time-pregnant animals). Estimates for animal requirements are based on the number of acutely isolated neurons that can typically be derived from one animal and the number of drugs that have been proposed to be investigated.

Compounds may be assessed in adult animals; ovariectomized or sham surgery treated; control or compound treated; and experiments may be performed in triplicate. For each of the six (two from each of the parent, the C-2 alkylation and the aminoethanol substitution compounds) maximally neuroprotective compounds 30 animals may be tested in neuroprotection studies. For each compound 5 points in a dose response curve may be assayed leading to a total anticipated animal requirement of 900.

Planar lipid bilayer single channel electrophysiology. ER vesicles or artificial liposomes containing ICCs may be incorporated into planar lipid bilayers and channel activity may be recorded electrophysiologically with the planar lipid bilayer being equivalent to the plasma membrane patch of patch clamp electrophysiology. Lipid bilayer membranes may be formed in a hole in a Teflon partition, which separates two buffer filled compartments (phosphatidylethanolamine and phosphatidylserine, 3:1 w/w, dissolved in decane, 40 mg lipid per ml, Avanti Polar Lipids, Alabaster, Ala.). Vesicles containing pc-2 may be added to one compartment (corresponding to the cytosolic compartment) and fuse with the bilayer leaving the channels contained in the vesicle as the only electrical connection between the two compartments. The buffer on the cytosolic side of the channel is a 250 mM HEPES-Tris solution, pH 7.35 and a 250 mM HEPES, 55 mM Ba(OH)₂ (or defined concentration of other current carrier, such as Ca(OH)₂ or Mg(OH)₂) solution, pH 7.35 constitutes the buffer on the trans side of the bilayer. Proper insertion of the ICCs may be monitored by appropriate stimulation of channels on the cytosolic side (IP₃R: Ca²⁺, IP₃; RyR: Ca²⁺, caffeine, cADPR; pc-2: Ca²⁺, activating membrane potential; 77, 83, 115-117, 141). Improperly inserted channels that are less than 5% under the described conditions will not be included in the analysis. Channel insertion and activity may be recorded with Ag/AgCl electrodes contacting the buffer filled compartments via KCl containing agarose bridges. Activity of a single channel protein can be monitored because it passes enough current to be measured electronically using an amplifier. Altering the divalent cation species on the luminal side of the channel that carries the current will test ion selectivity. Single channel conductance, voltage-dependence, dwell times and dependence on intracellular free Ca²⁺ concentrations may be determined using standard methods in the presence of blockers of other ICCs (IP₃R: no IP₃, heparin; RyR: ryanodine, ruthenium red; pc-2: lack of activating membrane potential; 77, 83). Modulating reagents may be added to the cytosolic side of the protein using a perfusion system (77, 83).

Optical imaging of intracellular Ca²⁺ concentrations/Ca²⁺ imaging. During the experiments, the cells may be kept in extracellular solution (ECS, in mM: NaCl, 137; KCl, 5; CaCl₂, 2; Na₂HPO₄, 1; MgSO₄, 1; HEPES, 10; glucose, 22; pH 7.4) in a perfusion chamber on the microscope stage at 37° C. and may be superfused continuously with ECS at a flow rate of 1 ml/minute. Cells may be incubated in ECS containing 2 μM cell permeant fluo-4 or fura-2 (fluo-4/fura-2 acetoxymethyl ester; Molecular Probes, Eugene, Oreg.) with 0.05% DMSO for 15-30 minutes and may be washed in ECS prior to the optical recording. Agonists and antagonists for cell surface receptors and membrane-permeable modulators may be bath-applied directly into the perfusion medium or with a micro puffer-pipette (115, 123-124). Identical volumes of ECS or water may be used as control applications. Ca²⁺ free ECS may be used to identify the contribution of intracellular and extracellular Ca²⁺ sources to Ca²⁺ transients. The fluorescence present in loaded cells may be measured with a Biorad confocal laser scanning microscope system equipped with a Zeiss Axiovert S100 (Zeiss, Oberkochen, Germany) or an imaging system for fast analysis of ratiometric dye measurements (Olympus IX-70, Olympus Corp., Japan; SimplePCI, C-imaging systems, Compix, Inc.).

The following assays may be used to assess neuronal viability and different stages of cell death. Optical assessment._Changes in cell morphology (apoptotic and necrotic morphology) and cell number may be used as an initial assessment of neurotoxicity and neuroprotection (see preliminary results in FIG. 8) using bright field differential interference contrast microscopy (Olympus IX-70, Olympus Corp., Japan; SimplePCI, C-imaging systems, Compix, Inc.).

Optical assessment using fluorescently labeled dyes. Viability of cells may be assessed with a Viability/Cytotoxicity kit for animal cells (Molecular Probes, Eugene Oreg.). This test specifically stains intact live cells and dead cells with damaged cellular membranes using cell-permeant und cell-impermeant fluorescent dyes. Cells are recognized easily as necrotic and may be quantified using the SimplePCI imaging software.

Fluorescence terminal deoxynucleotidyl transferase mediated X-dUTP nick-end labeling (TUNEL) cytochemistry. Cells having entered advanced stages of apoptosis may be detected with a TUNEL assay using the DeadEnd™ fluorometric TUNEL system kit (Promega, Madison, Wis.; fluorescein label) according to the manufacturer's instructions. The fluorescenly labeled cultures may be counter-stained with DAPI (Molecular Probes) to visualize the total number of cells.

Annexin V staining. The detection of Annexin V staining may be based upon the protocol provided in the Annexin V kit (Roche Molecular Biochemicals). The principal of the assay is based on the affinity of Annexin V, a calcium-dependent phospholipid binding protein, to bind with high affinity to phosphatidylserine. In healthy cells, Annexin V is unable to access the phosphatidylserine, which normally resides in the inner leaflet of the plasma membrane. However, during apoptotic cell death, phosphatidylserine becomes exposed at the outer surface of the membrane and is readily bound by Annexin V. Therefore, Annexin V immunostaining is an early marker for apoptotic cell death. Simultaneously, cultures may be treated with the plasma membrane impermeable nuclear stain, BOBO-1 (Molecular Probes, Inc.), serving as an index of necrotic cell death. BOBO-1 stains the nuclei of only those cells that have compromised membrane integrity (a hallmark of necrotic cell death). Using these markers of cell death, cells that are dually stained with Annexin V and BOBO-1 may be defined as necrotic. Those stained only with Annexin-V may be apoptotic, and those unstained may be designated as viable (live) cells. Cultures will first be washed with PBS (3×2 min), followed by incubation with Annexin-V (conjugated to the fluorophore, Alexa 568) and BOBO-1 for 15 min, washed 3×2 min with PBS, immediately analyzed for fluorescence using confocal microscopy. Staurosporine-treated cultures (1 μM for 3 hrs) will serve as the positive control for inducing apoptotic cell death.

LDH assay. This assay for cytotoxicity may be carried out according to the method provided in the Cytotoxicity Detection Assay kit (Roche Molecular Biochemicals). This colorimetric assay is based on the ability of LDH to promote the cleavage of a pale yellow tetrazolium salt to a water-soluble formazan dye product (red). 25 ml of media may be evaluated in triplicate, and the production of the dye product may be measured by evaluating the absorbance at 500 nm. Higher levels of LDH are indicative of greater cell death.

PARP cleavage. Poly ADP ribose Polymerase (PARP) is a 113 kD protein that is a substrate for such caspases as caspase 3 and 7. These proteases cleave PARP into fragments of about 89 kD and 24 kD. Using Western blot analysis, we will determine the relative amounts of the 89 kD cleavage product as compared to full length PARP. Differences in expression of these cleavage products may be indicative of the relative amount of caspase-mediated apoptosis.

Western Blot Analysis. Cells may be lysed in lysis buffer (50 mM Tris (pH 7.4), 150 mM NaCl, 10% glycerol, 1 mM EGTA, 1 mM Na3VO4, 5 μM ZnCl2, 100 mM NaF, 1% Triton X-100, 10 μg/ml aprotinin, 1 μg/ml leupeptin, and 1 mM PMSF) and snap frozen in liquid nitrogen. Following homogenization, by trituration of the cells through a 22 Ga needle, and centrifugation at 100,000×g for 15 min at 4° C., resulting supernatants (lysates) are normalized for protein content (Lowry Assay, Biorad DC Protein Assay Kit). 25 mg of protein from each sample will then be separated on a 10% polyacrylamide gel (SDS/PAGE) followed by transfer onto PVDF membranes (0.22 mm pore size, Biorad, Hercules, Calif.). The membranes are then blocked overnight with 3% BSA (Fraction V; Sigma) or in Tris-buffered saline containing 0.2% Tween-20 (TBS-T) and probed with the appropriate antibody. For ERK phosphorylation: rabbit anti-phosphoMAPK (dual phosphospecific (Thr202/Tyr204), 1:1000, Cell Signaling Technology, Beverly, Mass.); for ERK protein assessment: goat anti-ERK1 (1:1000); goat anti-ERK2 (1:1000, Santa Cruz Biotechnologies, Santa Cruz, Calif.). For Akt phosphorylation, rabbit anti-phosphoAkt (Ser473) or rabbit anti-phosphoAkt(Thr308) (Cell Signaling Technology); for Akt protein assessment, rabbit anti-Akt (New England Biolabs). Binding of the primary antibody to the membrane may be detected using a secondary antibody (either goat anti-rabbit or goat anti-mouse), conjugated to horseradish peroxidase (HRP; 1:40,000, Pierce, Rockford, Ill.) and visualized using enzyme-linked chemiluminescence (ECL; Amersham, Arlington Heights, Ill.). All blots may be re-probed to verify equal loading of protein across lanes. For the assessment of Bcl-2:Bax ratios, band intensity signals may be quantified using standard densitometry software and calibration by loading controls (Labworks Image software, UVP Imaging systems, CA).

TABLE 2
Antibodies directed against signaling proteins used for the proposed
experiments. Specific antibodies have been tested with regard
to epitope specificity and their experimental protocol parameters
(fixation time of the tissue, dilution, secondary antibodies)
have been established and optimized in the laboratory (mAb: monoclonal
antibody; pAb polyclonal antiserum).

| Antibodies | | Antibody | |
|---|---|---|---|
| directed against | source | type/host | dilution |
| PhosphoERK | Cell Signaling Technologies | mAb, mouse | 1:500 |
| (T202/Y204) | | | |
| Goat anti- | Santa Cruz Biotechnology | pAb, goat | 1:2000 |
| ERK1 (C-16) | | | |
| Goat anti- | Santa Cruz Biotechnology | pAb, goat | 1:2000 |
| ERK2 (C-14) | | | |
| B-Raf | Upstate Biotechnology | mAb, mouse | 1:200 |
| c-Raf | Upstate Biotechnology | mAb, mouse | 1:200 |
| MEK1 (C-18) | Santa Cruz Biotechnology | mAb, mouse | 1:100 |
| MEK2 | Transduction labs | pAb, rabbit | 1:1000 |
| PhosphoAkt | Cell Signaling Technologies | mAb, mouse | 1:200 |
| (Ser473) | | | |
| PhosphoAkt | Cell Signaling Technologies | mAb, mouse | 1:100 |
| (Thr308) | | | |
| Akt | Cell Signaling Technologies | pAb, rabbit | 1:1000 |
| Bax | Cell Signaling Technologies | pAb, rabbit | 1:1000 |
| Bcl-x(L) | Cell Signaling Technologies | pAb, rabbit | 1:1000 |

In vitro kinase assays. Cells may be lysed and processed as described above. Approximately 400 μg of sample lysate is incubated with a rabbit anti B-Raf (UBI, Lake Placid, N.Y.) or rabbit anti c-Raf antibody (UBI) and precipitated using anti-rabbit IgG-coated magnetic beads (Dynabeads, Dynal A. S., Oslo, Norway). Following 4 washes with lysis buffer, the B-Raf or C-Raf captured beads are used as the starting material for the kinase assay. The assay procedure is done according to the protocol provided in the Raf kinase assay kit (UBI) and is based on the phosphorylation of myelin basic protein (MBP) by a Raf-activated kinase cascade, using radioactive ATP as the final phosphate donor. Assay dilution buffer (20 mM MOPS, pH 7.2, 25 mM β-glycerol phosphate, 5 mM EGTA, 1 mM Na₃VO₄, 1 mM dithiothreitol) and a Mg²⁺/cold ATP cocktail are added in conjunction with 0.4 μg of inactive MEK1 and 1 μg of inactive GST-p42 MAPK. This mixture is then incubated for 30 min at 30° C. Subsequently, additional assay dilution buffer, MBP and [γ-³²P] ATP are added and incubated for an additional 10 min at 30° C. while shaking vigorously. After boiling the samples for 5 min, 25 ml of the supernatant is spotted onto P81 phosphocellulose paper, which exhibits differential binding of the phosphorylated MBP from unincorporated ³²P. Radioactivity incorporated into the P81 paper will then be counted using a scintillation counter. For evaluation of the kinase activities for MEK and ERK, the same general procedure may be employed. In all kinase assays, parallel methodological controls (pre-immune IgG) may be performed for non-specific kinase activity.

Cell Culture. Primary neuronal cells from hippocampus tissue may be obtained from postnatal day (P) 2 animals. Neurons may be isolated enzymatically and mechanically and may be maintained on rat-tail collagen-coated/poly-D-lysine pre-coated glass coverslips and grown in steroid-deficient and phenol red-free maintenance medium [gelding serum (25%); Hank's BSS (22.5%); EME (50%); glucose (7.5 mg/ml); L-glutamine (2 mM); ascorbic acid (50 μg/ml)]. The cultures may be maintained in vitro for 7 days prior to the application of the appropriate treatment with a specified dose or for a designated length of time. Control cultures will always be sham treated to account for any consequences of procedural manipulation.

Western Blot Analysis. Cells may be placed into lysis buffer (50 mM Tris (pH 7.4), 150 mM NaCl, 10% glycerol, 1 mM EGTA, 1 mM Na3VO4, 5 μM ZnCl2, 100 mM NaF, 1% Triton X-100, 10 μg/ml aprotinin, 1 μg/ml leupeptin, and 1 mM PMSF) and snap frozen in liquid nitrogen. Following homogenization, by trituration of the tissue through a 22 Ga needle, and centrifugation at 100,000×g for 15 min at 4° C., resulting supernatants (lysates) are normalized for protein content (Lowry Assay, Biorad DC Protein Assay Kit). 25 mg of protein from each sample will then be separated on a 10% polyacrylamide gel (SDS/PAGE) followed by transfer onto PVDF membranes (0.22 mm pore size, Biorad, Hercules, Calif.). The membranes are then blocked overnight with 3% BSA (Fraction V; Sigma) or in Tris-buffered saline containing 0.2% Tween-20 (TBS-T) and probed with the appropriate antibody (table 2). Binding of the primary antibody to the membrane may be detected using a secondary antibody (either goat anti-rabbit or goat anti-mouse), conjugated to horseradish peroxidase (HRP; 1:40,000, Pierce, Rockford, Ill.) and visualized on autoradiographic film, using enzyme-linked chemiluminescence (ECL; Amersham, Arlington Heights, Ill.). All blots may be re-probed with the appropriate antibody to verify equal loading of protein across lanes.

Immunohistochemistry. Immediately following treatment, the cultures (which are still attached to collagen-coated coverslips) are rinsed briefly with phosphate buffered saline, and fixed in 4% paraformaldehyde for 1 hr at 4° C. After 2 rinses with ice cold PBS, the cultures are then incubated on a rocking platform with blocking solution (10% donkey serum/5% non-fat dry milk in Tris-buffered saline containing 0.5% Triton X-100 (TBS-T)). After blocking, the cultures are incubated with the primary antibodies (table 2), followed by several washes (with TBS-T) and subsequent re-blocking. Then, the incubation with the secondary antibody (bridging antibody: goat anti-rabbit), together with the mouse anti-MAP-2B (Roche Molecular Diagnostics, Indianapolis, Ind.) antibody (specifically identifies neurons) is carried out, followed again by thorough washing, and subsequent re-blocking. Next, the fluorophore-conjugated antibodies, Cyanine-3 coupled donkey anti-goat (Jackson Immunoresearch labs, West Grove, Pa.) antibody (which will react with the bridging antibody) and Cyanine-5 coupled donkey anti-mouse (Jackson Immunoresearch labs) antibody (which will react with the MAP-2B antibody), are added to the cultures and incubated. After washes with TBS-T, the cultures are then incubated with the nuclear dye (Sytox, Molecular Probes) for 15 min and subsequently washed with TBS. The cultures are then mounted onto glass slides using Vectashield mounting media and viewed under a Confocal Laser Scanning Microscope (core facility, UNTHSC).

Animals and Ovariectomy. Bilateral ovariectomy may be performed on female Charles Rivers rats weighing 200-225 g under methoxyflurane (Metophane®, Pitman Moore, Crossings, N.J.) inhalant anesthesia 14 days prior to drug administration and MCA occlusion.

NAE Treatments. NAE may be dissolved in, e.g., corn oil and the solution placed in Silastic® tubes (Dow-Corning, Midland, Mich.) that are closed on either end with Silastic Medical Adhesive® (Dow-Corning). Sham (oil containing) pellets may be similarly prepared, but with addition of the oil vehicle only. All pellets were washed with methanol to remove steroid adhering to the outside. Subsequently, pellets may be washed in physiological saline, a procedure that assures first order in vivo release of NAEs to achieve physiologically relevant concentrations. The pellets may be implanted subcutaneously (sc) into ovariectomized rats at various times prior to the MCA occlusion.

Cerebral Ischemia. At 14 days after ovariectomy, animals may be anesthetized with ketamine (60 mg/kg, i.p.) and xylazine (10 mg/kg, ip). During surgery, rectal temperature is monitored and body temperature is adjusted with a heating lamp, to maintain rectal temperature between 36.5 and 37.0° C. After the surgery, body temperature is maintained at an environmental temperature of 37° C. Using an operating microscope, the left carotid artery is exposed through a midline incision of the neck skin. The sternohyloid, digastic (posterior belly) and the omohyloid muscles are divided and retracted. Then the greater horn of the hyloid bone is removed for exposure of the distal external carotid artery (ECA). The common carotid artery (CCA) is dissected from the vagus nerve and the ECA and its branches (occipital and superior thyroid arteries) are dissected distally. The internal carotid artery (ICA) is carefully separated from the vagus and glossopharyngeal nerves just below the ECA. Near the base of the skull, the ICA has an extracranial branch, the pterygopalatine artery. Beyond this bifurcation, the ICA enters the cranium medially. After the arteries and their branches are dissected, the distal ECA and its branches, the CCA and the pterygopalatine arteries are cauterized completely. The ECA and the occipital arteries are cut, and then a microvascular clip is placed on the ICA near the base of the skull. The tip of a 2.5 cm length of 3-0 monofilament nylon suture is heated to create a globule for easy movement and blockade of the lumen of the vessel. The suture is introduced into the ECA lumen through a puncture and was gently advanced to the distal ICA until it reached the clipped position. The microvascular clip is then removed and the suture was inserted until resistance was felt. The resistance indicated that the suture has passed the middle cerebral artery origin and reached the proximal segment of the anterior cerebral artery. This operative procedure is completed within 10 min without bleeding. After the prescribed occlusion time (1 hour), the suture is withdrawn from the ICA and the distal ICA is immediately cauterized. Initial assessment of neuroprotection will use 2 mm thick coronal sections and staining with 2% 2,3,5-Triphenyl-2H-tetrazolium chloride (TTC) in a 0.9% saline solution at 37° C. for 30 min followed by fixation in 10% formalin (see FIG. 7).

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

References

1. Chapman, K. D 1998. Phospholipase activity in plant growth and development and in response to environmental stress. Trends in Plant Science 3: 319-426.
2. Munnik, T., Irvine, R. F., Musgrave, A. (1998) Phospholipid signalling in plants. Biochim. Biophys. Acta 1389: 222-272.
3. Schmid, H. H. O., Schmid, P. C., Natarajan, V. 1996. The N-acylation-phosphodiesterase pathway and cell signalling. Chem. Phys. Lipid. 80: 133-142.
4. Devane, W. A., Hanus, L., Brewer, A., Pertwee, R. G., Steveson, L. A., Griffin, G, Gibson, D. Mandelbaum, A. Etinger, A., Mechoulam, R. (1992) Isolation and structure of a brain constituent that binds to the cannabinoid receptor. Science 258: 1946-1949.
5. Chapman, K. D., Venables, B. J., Markovic, R. M., Blair, R. Betinger, C. (1999) N-Acylethanolamines in seeds: quantification of molecular species and their degradation upon imbibition. Plant Physiol. 120: 1157-1164
6. Chapman K. D., B. J. Venables, R. Blair, Jr., and C. Bettinger. N-Acylethanolamines in Seeds: Quantification of Molecular Species and their Degradation upon Imbibition, Plant Physiol120: 1157-1164 (1999).
7. Di Tomaso E, M. Beltramo, D. Piomelli, Brain Cannabinoids in Chocolate, Nature 382(6593): 677-8 (1996).
8. DiMarzo V., N. Sepe, L. DePetrocellis, A. Berger, G. Crozier, E. Fride, R. Mechoulam, Trick or Treat from Food Endocannabinoids, Nature 396(6712): 636-637 (1998).
9. Shrestha R., M. Noordimeer, M. van der Stelt, G. Veldink, and K. D. Chapman, N-Acylethanolamines Are Metabolized by Lipoxygenase and Amidohydrolase in Two Competing Pathways during Cotton (Gossypium hirsutum L) Seed Imbibition. Plant Physiol, First published on Aug. 8, 2002 on-line in advance of hard copy; 10. 1104/pp.004689 (2002).
10. DiMarzo, V. (1998) Endocannabinoids and other fatty acid derivatives with cannabimimetic properties: biochemistry and possible physiopathological relevance. Biochim.Biophys.Acta 1392: 153-175.
11. Schmid H. H. O., P. C. Schmid, V. Natarajan, N-Acylated Glycerophospholipids and their Derivatives, Prog. Lipid Res. 29(1): 1-43 (1990).
12. Chapman K. D., Emerging Physiological Roles for N-Acylphosphatidylethanolamine Metabolism in Plants: Signal Transduction and Membrane Protection, Chern. Phys. Lipids 108(1-2):221-229 (2000).
13. Howlett A. C., F. Barth, T. I. Bonner., G. Cabral, P. Casellas, W. A. Devane, C. C. Felder, M. Herkenham, K. Mackie, B. R. Martin, R. Mechoulam, and R. G. Pertwee, International Union of Pharmacology. XXVII. Classification of Cannabinoid Receptors, Pharmacal. Rev. 54(2):161-202 (2002).
14. Di Marzo V., L. De Petrocellis, F. Fezza, A. Ligresti, and T. Bisogno, Anandamide Receptors. Prostaglandins Leukot. Essent. Fatty Acids. 66(2-3): 377-391 (2002).
15. Cravatt B. F., K. Demarest, M. P. Patricelli, M. H. Bracey, D. K. Giang, B. R. Martin, and A. H. Lichtman, Supersensitivity to Anandamide and Enhanced Endogenous Cannabinoid Signaling in Mice Lacking Fatty Acid Amide Hydrolase, Proc. Natl. Acad. Sci. USA 98: 9371-9376 (2001).
16. Zimmer A, A. Zimmer, A. G. Hohmann, M. Herkenham, and T. Bonner, Increased Mortality, Hypoactivity, and Hypoalgesia in Cannabinoid CB1 Receptor Knockout Mice. Proc Natl Acad Sci USA 96: 5780-5785 (1999).
17. Wilson R. I., and R. A. Nicoll, Endocannabinoid Signaling in the Brain. Science 296: 678-682 (2002).
18. Beltramo, M., Stella, N, Calignano, A. Lin, S. Y., Makriyannis, A., Piomelli, D. 1997. Functional role of high-affinity anandamide transport, as revealed by selective inhibition. Science 277: 1094-1097.
19. Cravatt, B. F., Giang, D. K., Mayfield, S. P., Boger, D. L., Lerner, R. A., Gilula, N. B. 1996. Molecular characterization of an enzyme that degrades neuromodulatory fatty acid amides. Nature (London) 384: 83-87.
20. Maccarrone, M., van der Stelt, M, Rossi, A, Veldink, G. A., Vliegenthart, J. F. G., Agro, A. F. (1998) Anandamide hydrolysis by human cells in culture and brain. J. Biol. Chem. 273: 32332-32339.
21. Calignano, A. LaRana, G. Giuffrida, A., Piomelli, D (1998) Control of Pain initiation by endogenous cannabinoids. Nature: 394:277-280.
22. Pessah I. N., Francini A. O., Scales D. J., Waterhouse A. L., and Casida J. E. Calcium-ryanodine receptor complex. Solubilization and partial characterization from skeletal muscle junctional sarcoplasmic reticulum vesicles. (1986) J. Biol. Chem. 261(19),8643-8648.
23. Imagawa T., Smith J. S., Coronado R., and Campbell K. P. Purified ryanodine receptor from skeletal muscle sarcoplasmic reticulum is the Ca2+-permeable pore of the calcium release channel. (1987) J. Biol. Chem. 262(34), 16636-16643.
24. Smith J. S., Imagawa T., Ma J., Fill M., Campbell K. P., and Coronado R. Purified ryanodine receptor from rabbit skeletal muscle is the calcium-release channel of sarcoplasmic reticulum. (1988) J. Gen. Physiol. 92(1), 1-26.
25. Lai F. A., Anderson K., Rousseau E., Liu Q. Y., and Meissner G. Evidence for a Ca2+ channel within the ryanodine receptor complex from cardiac sarcoplasmic reticulum. (1988) Biochem. Biophys. Res. Commun. 151(1), 441-449.
26. Hymel L., Inui M., Fleischer S., and Schindler H. Purified ryanodine receptor of skeletal muscle sarcoplasmic reticulum forms Ca2+-activated oligomeric Ca2+ channels in planar bilayers. (1988) Proc. Natl. Acad. Sci. USA. 85(2), 441-445.
27. Hymel L., Schindler H., Inui M., and Fleischer S. Reconstitution of purified cardiac muscle calcium release channel (ryanodine receptor) in planar bilayers. (1988) Biochem. Biophys. Res. Commun. 152(1), 308-314.
28. Mignery, G. A., Südhof, T. C., Takei, K., and De Camilli, P. Putative receptor for inositol 1,4,5-trisphosphate similar to ryanodine receptor. (1989) Nature 342, 192-195.
29. Walton P. D., Airey J. A., Sutko J. L., Beck C. F., Mignery G. A., Südhof T. C., Deerinck T. J., and Ellisman M. H. Ryanodine and inositol trisphosphate receptors coexist in avian cerebellar Purkinje neurons. (1991) J. Cell Biol. 113(5), 1145-1157.
30. Takei K., Stukenbrok H., Metcalf A., Mignery G. A., Sudhof T. C., Volpe P., and De Camilli P. Ca2+ stores in Purkinje neurons: endoplasmic reticulum subcompartments demonstrated by the heterogeneous distribution of the InsP3 receptor, Ca(2+)-ATPase, and calsequestrin. (1992) J. Neurosci. 12(2), 489-505.
31. Berridge M. J. Neuronal calcium signaling. (1998) Neuron. 21(1), 13-26.
32. Augustine G. J., Charlton M. P., and Smith S. J. Calcium action in synaptic transmitter release. (1987) Annu Rev. Neurosci. 10, 633-693.
33. Smith S. J., Augustine G. J. Calcium ions, active zones and synaptic transmitter release. (1988) Trends Neurosci. 11(10), 458-464.
34. Augustine G. J., Adler E. M., and Charlton M. P. The calcium signal for transmitter secretion from presynaptic nerve terminals. (1991) Ann. N. Y. Acad. Sci. 635, 365-381.
35. Augustine G. J., and Neher E. Neuronal Ca2+ signaling takes the local route. (1992) Curr. Opin. Neurobiol. 2(3), 302-307.
36. Tempia F., and Konnerth A. Calcium requirement of long-term depression and rebound potentiation in cerebellar Purkinje neurons. (1994) Semin. Cell Biol. 5(4), 243-250.
37. Eilers J., Plant T., and Konnerth A. Localized calcium signaling and neuronal integration in cerebellar Purkinje neurones. (1996) Cell Calcium. 20(2), 215-226.
38. Eilers J., and Konnerth A. Dendritic signal integration. (1997) Curr. Opin. Neurobiol. 7(3), 385-390.
39. Taylor C. W. Inositol trisphosphate receptors: Ca2+-modulated intracellular Ca2+ channels. (1998) Biochim. Biophys. Acta. 1436(1-2), 19-33.
40. Taylor C. W., Genazzani A. A., and Morris S. A. Expression of inositol trisphosphate receptors. (1999) Cell Calcium. 26(6), 237-251.
41. Patel S., Joseph S. K., and Thomas A. P. Molecular properties of inositol 1,4,5-trisphosphate receptors. (1999) Cell Calcium. 25(3), 247-264.
42. Sorrentino V., Barone V., and Rossi D. Intracellular Ca(2+) release channels in evolution. (2000) Curr. Opin. Genet. Dev. 10(6), 662-667.
43. Ehrlich B. E. Functional properties of intracellular calcium-release channels. (1995) Curr. Opin. Neurobiol. 5(3), 304-309.
44. Striggow F., and Ehrlich B. E. Regulation of intracellular calcium release channel function by arachidonic acid and leukotriene B4. (1997) Biochem. Biophys. Res. Commun. 237(2), 413-418.
45. Xu L., Tripathy A., Pasek D. A., and Meissner G. Potential for pharmacology of ryanodine receptor/calcium release channels. (1998) Ann. N. Y. Acad. Sci. 853, 130-148.
46. Guse A. H. Cyclic ADP-ribose. (2000) J. Mol. Med. 78(1), 26-35.
47. Bezprozvanny I., Watras J., and Ehrlich B. E. Bell-shaped calcium-response curves of IP3- and calcium-gated channels from endoplasmic reticulum of cerebellum. (1991) Nature. 351(6329), 751-754.
48. Meissner G., Rios E., Tripathy A., Pasek D. A. Regulation of skeletal muscle Ca2+ release channel (ryanodine receptor) by Ca2+ and monovalent cations and anions. (1997) J. Biol. Chem. 272(3), 1628-1638.
49. Bezprozvanny I., and Ehrlich B. E. ATP modulates the function of inositol 1,4,5-trisphosphate-gated channels at two sites. (1993) Neuron. 10(6), 1175-1184.
50. Thrower E. C., Mobasheri H., Dargan S., Marius P., Lea E. J., Dawson A. P. Interaction of luminal calcium and cytosolic ATP in the control of type 1 inositol (1,4,5)-trisphosphate receptor channels. (2000) J. Biol. Chem. 275(46), 36049-36055.
51. Berridge, M. J. Rapid accumulation of inositol-trisphosphate reveals that agonists hydrolyse polyphosphoinositides instead of phosphatidylinositol. (1983) Biochem. J. 212, 849-858.
52. Mignery, G. A., Newton, C. L., Archer, B. T. III, and Südhof, T. C. Structure and expression of the rat inositol 1,4,5-trisphosphate receptor. (1990) J. Biol. Chem. 265, 12679-12685.
53. Miyawaki, A., Furuichi, T., Ryou, Y., Yoshikawa, S., Nakagawa, T., Saitoh, T., and Mikoshiba, K. Structure-function relationships of the mouse inositol 1,4,5-trisphosphate receptor. (1991) Proc. Natl. Acad. Sci. USA 88, 4911-4915.
54. Yoshikawa, F, H Iwasaki, T Michikawa, T Furuichi, and K Mikoshiba. Cooperative formation of the ligand-binding site of the inositol 1,4,5-trisphosphate receptor by two separable domains. (1999) J. Biol. Chem. 274, 328-34.
55. Furuichi, T., Yoshikawa, S., and Mikoshiba, K. Nucleotide sequence of cDNA encoding P400 protein in the mouse cerebellum. (1989) Nucl. Acids. Res. 17, 5385-5386.
56. Furuichi, T., Yoshikawa, S., Miyawaki, A., Wada, K., Maeda, N., and Mikoshiba, K. Primary structure and functional expression of the inositol 1,4,5-trisphosphate-binding protein P400. (1989) Nature 342, 32-38.
57. Maeda, N., Kawasaki, T., Nakade, S., Yokota, N., Taguchi, T., Kasai, M., and Mikoshiba, K. Structural and funcional characterization of inositol (1,4,5) trisphosphate receptor channel from mouse cerebellum. (1991) J. Biol. Chem. 266, 1109-1116.
58. Sienaert, I, DeSmedt, H, Parys, J. B., Missiaen, L, VanLingen, S., Sipma, H., and Casteels R. Characterization of a cytosolic and a luminal Ca2+ binding site in the type I inositol 1,4,5-trisphosphate receptor. (1996) J. Biol. Chem. 271, 27005-27012.
59. Sienaert, I, L Missiaen, H De Smedt, J Parys, H Sipma, and R Casteels. Molecular and functional evidence for multiple Ca2+-binding domains in the type I inositol 1,4,5-trisphosphate receptor. (1997) J. Biol. Chem. 272, 25899-25906.
60. Michikawa, T, J Hirota, S Kawano, M Hiraoka, M Yamada, T Furuichi, and K Mikoshiba. Calmodulin mediates calcium-dependent inactivation of the cerebellar type 1 inositol 1,4,5-trisphosphate receptor. (1999) Neuron. 23, 799-808.
61. Südhof, T. C., Newton, C. L., Archer, B. T. III, Ushkaryov, Y. A., and Mignery, G. A. Structure of a novel InsP3 receptor. (1991) EMBO J. 10, 3199-3206.
62. Blondel, O, J Takeda, H Janssen, S Seino, and G I Bell. Sequence and functional characterization of a third inositol trisphosphate receptor subtype, IP3R-3, expressed in pancreatic islets, gastrointestinal tract, and other tissues. (1993) J. Biol. Chem. 268, 11356-11363.
63. Maranto, A R. Primary structure, ligand binding, and localization of the human type 3 inositol 1,4,5-trisphosphate receptor expressed in intestinal epithelium. (1994) J. Biol. Chem. 269, 1222-1230.
64. Bush, K T, R O Stuart, S H Li, L A Moura, A H Sharp, C A Ross, and S K Nigam. Epithelial inositol 1,4,5-trisphosphate receptors: multiplicity of localization, solubility, and isoforms. (1994) J. Biol. Chem. 269, 23694-23699.
65. Nathanson, M. H., M. B. Fallon, P. J. Padfield, and A. R. Maranto. Localization of the type 3 inositol 1,4,5-trisphosphate receptor in the Ca2+ wave trigger zone of the pancreatic acinar cells. (1994) J. Biol. Chem. 269, 4693-4696.
66. Ramos-Franco, J, M Fill, and G A Mignery. Isoform-specific function of single inositol 1,4,5-trisphosphate receptor channels. (1998) Biophys J. 75, 834-839.
67. Hagar, R, A Burgstahler, M Nathanson, and B Ehrlich. Type III InsP3 receptors stay open in the presence of increased calcium. (1998) Nature 396, 81-84.
68. Smith J. S, Coronado R., and Meissner G. Single channel measurements of the calcium release channel from skeletal muscle sarcoplasmic reticulum. Activation by Ca2+ and ATP and modulation by Mg2+. (1986) J. Gen. Physiol. 88(5), 573-588.
69. Ehrlich B. E., and Watras J. Inositol 1,4,5-trisphosphate activates a channel from smooth muscle sarcoplasmic reticulum. (1988) Nature. 336(6199), 583-586.
70. Supattapone S., Worley P. F., Baraban J. M., and Snyder S. H. Solubilization, purification, and characterization of an inositol trisphosphate receptor. (1988) J. Biol. Chem. 263, 1530-1534.
71. Palade P., Dettbarn C., Alderson B., and Volpe P. Pharmacologic differentiation between inositol-1,4,5-trisphosphate-induced Ca2+ release and Ca2+- or caffeine-induced Ca2+ release from intracellular membrane systems. (1989) Mol. Pharmacol. 36(4), 673-680.
72. MacKrill J. J. Protein-protein interactions in intracellular Ca2+-release channel function. (1999) Biochem. J. 337 (Pt 3), 345-361.
73. Sabbadini R. A., Betto R., Teresi A., Fachechi-Cassano G., and Salviati G. The effects of sphingosine on sarcoplasmic reticulum membrane calcium release. (1992) J. Biol. Chem. 267(22), 15475-15484.
74. Needleman D. H., Aghdasi B., Seryshev A. B., Schroepfer G. J., and Hamilton S. L. Modulation of skeletal muscle Ca2(+)-release channel activity by sphingosine. (1997) Am. J. Physiol. 272(5 Pt 1), C1465-C1474.
75. Sharma C., Smith T., Li S., Schroepfer G. J., and Needleman D. H. Inhibition of Ca2+ release channel (ryanodine receptor) activity by sphingolipid bases: mechanism of action. (2000) Chem. Phys. Lipids. 104(1), 1-11.
76. Smith J. S., Coronado R., and Meissner G. Sarcoplasmic reticulum contains adenine nucleotide-activated calcium channels. (1985) Nature. 316(6027), 446-449.
77. Koulen, P., Cai, Y., Maeda, Y., Somlo, S., and Ehrlich, B. Polycystin-2—A divalent Cation channel of the endoplasmic reticulum. (2000) J Am Soc Nephrol. 11, 393A.
78. Berridge M J. Elementary and global aspects of calcium signaling. (1997) J Physiol., 499, 291-306.
79. Mochizuki T, Wu G, Hayashi T, Xenophontos S L, Veldhuisen B, Saris J J, Reynolds D M, Cai Y, Gabow P A, Pierides A, Kimberling W J, Breuning M H, Deltas C C, Peters D J, Somlo S. PKD2, a gene for polycystic kidney disease that encodes an integral membrane protein (1996). Science. 272, 1339-42.
80. Wu G, D'Agati V, Cai Y, Markowitz G, Park J H, Reynolds D M, Maeda Y, Le T C, Hou H Jr, Kucherlapati R, Edelmann W, Somlo S. Somatic inactivation of Pkd2 results in polycystic kidney disease. (1998) Cell. 93, 177-188.
81. Gonzalez-Perret S, Kim K, Ibarra C, Damiano A E, Zotta E, Batelli M, Harris P C, Reisin I L, Arnaout M A, Cantiello H F. Polycystin-2, the protein mutated in autosomal dominant polycystic kidney disease (ADPKD), is a Ca2+-permeable nonselective cation channel. (2001) Proc Natl Acad Sci USA. 98, 1182-1187.
82. Somlo S, Ehrlich B. Human disease: calcium signaling in polycystic kidney disease. (2001) Curr Biol. 11, R356-360.
83. Koulen P, Cai Y, Geng L, Maeda Y, Nishimura S, Witzgall R, Ehrlich B E, Somlo S. Polycystin-2 is an intracellular calcium release channel. Nat Cell Biol. 2002 March;4(3):191-197.
84. Greene, L. A. and Tischler A. S., Establishment of a noradrenergic clonal line of rat adrenal pheochromocytoma cells which respond to nerve growth factor, Proc Natl Acad Sci USA, 73 (1976) 2424-8.
85. Davis, R. J., Transcriptional regulation by MAP kinases, Mol Reprod Dev, 42 (1995) 459-67.
86. Marshall, C. J., MAP kinase kinase kinase, MAP kinase kinase and MAP kinase, Curr Opin Genet Dev, 4 (1994) 82-9.
87. Marshall, C., Specificity of receptor tyrosine kinase signaling: transient versus sustained extracellular signal-regulated kinase activation., Cell, 80 (1995) 179-85.
88. Qui, M. S. and Green S. H., PC12 cell neuronal differentiation is associated with prolonged p21ras activity and consequent prolonged ERK activity, Neuron, 9 (1992) 705-17.
89. Yan, C. Y. I. and Greene L. A., Prevention of PC12 cell death by N-acetylcysteine requires activation of the Ras pathway, J Neurosci, 18 (1998) 4042-9.Zhang L, Rubinow D R, Xaing G, Li B S, Chang Y H, Maric D, Barker J L, Ma W., Estrogen protects against beta-amyloid-induced neurotoxicity in rat hippocampal neurons by activation of Akt. Neuroreport. 12 (2001) 1919-23.
90. Creedon, D. J., Johnson E. M. and Lawrence J. C., Mitogen-activated protein kinase-independent pathways mediate the effects of nerve growth factor and cAMP on neuronal survival, J Biol Chem, 271 (1996) 20713-8.
91. Klesse, L. J. and Parada L. F., p21 ras and phosphatidylinositol-3 kinase are required for survival of wild-type and NF1 mutant sensory neurons, J Neurosci, 18 (1998) 10420-8.
92. Anderson, C. N. G. and Tolkovsky A. M., A role for MAPK/ERK in sympathetic neuron survival: protection against a p53-dependent, JNK-independent induction of apoptosis by cytosine arabinoside, J Neurosci, 19 (1999) 664-73.
93. Desdouits-Magnen, J., Desdouits F., Takeda S., Syu L. J., Saltiel A. R., Buxbaum J. D., Czernik A. J., Nairn A. C. and Greengard P., Regulation of secretion of Alzheimer amyloid precursor protein by the mitogen-activated protein kinase cascade, J Neurochem, 70 (1998) 524-30.
94. Mills, J., Laurent Charest D., Lam F., Beyreuther K., Ida N., Pelech S. L. and Reiner P. B., Regulation of amyloid precursor protein catabolism involves the mitogen-activated protein kinase signal transduction pathway, J Neurosci, 17 (1997) 9415-22.
95. Masters, C. L., Simms G., Weinman N. A., Multhaup G., McDonald B. L. and Beyreuther K., Amyloid plaque core protein in Alzheimer disease and Down syndrome, PNAS, 82 (1985) 4245-9.
96. Selkoe, D. J., Abraham C. R., Podlisny M. B. and Duffy L. K., Isolation of low-molecular-weight proteins from amyloid plaque fibers in Alzheimer's disease, J Neurochem, 46 (1986) 1820-34.
97. Carpenter, C. L. and Cantley L. C., Phosphoinositide kinases, Curr Opin Cell Biol, 8 (1996) 153-8.
98. Franke, T. F., Kaplan D. R. and Cantley L. C., P13K: downstream AKTion blocks apoptosis, Cell, 88 (1997) 435-7.
99. Franke, T. F., Kaplan D. R., Cantley L. C. and Toker A., Direct regulation of the Akt proto-oncogene product by phosphatidylinositol-3,4-bisphosphate, Science, 275 (1997) 665-8.
100. Dudek, H., Datta S. R., Franke T. F., Birnbaum M. J., Yao R., Cooper G. M., Segal R. A., Kaplan D. R. and Greenberg M. E., Regulation of neuronal survival by the serine-threonine protein kinase Akt, Science, 275 (1997) 661-5.
101. D'Mello, S. R., Borodezt K. and Soltoff S. P., Insulin-like growth factor and potassium depolarization maintain neuronal survival by distinct pathways: possible involvement of PI 3- kinase in IGF-1 signaling, J Neurosci, 17 (1997) 1548-60.
102. Zubenko, G. S., Stiffler J. S., Hughes H. B. and Martinez A. J., Reductions in brain phosphatidylinositol kinase activities in Alzheimer's disease, Biol Psychiatry, 45 (1999) 731-6.
103. Nethrapalli I S, Singh M, Guan X, Guo Q, Lubahn D B, Korach K S, Toran-Allerand C D., Estradiol (E2) elicits SRC phosphorylation in the mouse neocortex: the initial event in E2 activation of the MAPK cascade? Endocrinology 142 (2001) 5145-8.
104. Singh, M., Setalo Jr. G., Guan X. P., Warren M. and Toran-Allerand C. D., Estrogen-induced Activation of Mitogen-Activated Protein Kinase in Cerebral Cortical Explants: Convergence of Estrogen and Neurotrophin Signaling Pathways, J Neurosci, 19 (1999) 1179-1188.
105. Sétáló G Jr, Singh M, Guan X, Toran-Allerand C D., Estradiol-induced phosphorylation of ERK½ in explants of the mouse cerebral cortex: The roles of heat shock protein 90 (Hsp90) and MEK2. J Neurobiol 50 (2002) 1-12.
106. Singh M, Setalo G Jr, Guan X, Frail D E, Toran-Allerand C D., Estrogen-induced activation of the mitogen-activated protein kinase cascade in the cerebral cortex of estrogen receptor-alpha knock-out mice. J Neurosci 20 (2000) 1694-700.
107. Singer C A, Figueroa-Masot X A, Batchelor R H, Dorsa D M., The mitogen-activated protein kinase pathway mediates estrogen neuroprotection after glutamate toxicity in primary cortical neurons. J Neurosci. 19 (1999) 2455-63.
108. Singh, M, Ovarian hormones elicit phosphorylation of Akt and extracellular-signal regulated kinase in explants of the cerebral cortex. Endocrine 14 (2001) 407-15.
109. Honda K, Sawada H, Kihara T, Urushitani M, Nakamizo T, Akaike A, Shimohama S., Phosphatidylinositol 3-kinase mediates neuroprotection by estrogen in cultured cortical neurons. J Neurosci Res. 60 (2000) 321-7.
110. Honda K, Shimohama S, Sawada H, Kihara T, Nakamizo T, Shibasaki H, Akaike A., Nongenomic antiapoptotic signal transduction by estrogen in cultured cortical neurons. J Neurosci Res. 64:466-75, 2001.
111. Singer, C. A., Rogers K. L., Strickland T. M. and Dorsa D. M., Estrogen protects primary cortical neurons from glutamate toxicity, Neurosci Lett, 212 (1996) 13-6.
112. Chapman, K. D., Tripatby, S., Venables, B. J., Desouza, A. D. 1998. N-Acylethanolamines: fonnation and molecular composition of a new class of plant lipids. Plant Physiol. 116: 1163-1168.
113. Tripathy S, Venables, B. Chapman, K. D. (1999) N-Acylethanolamines in elicitor signal transduction. Plant Physiol. 121: 1299-1308.
114. Stella, N. Schweitzer, P., Piomelli, D. 1997. A second endogenous cannabinoid that modulates long-term potentiaition. Nature (London) 388: 773-778.
115. Koulen P., and Ehrlich B. E. Reversible block of the calcium release channel/ryanodine receptor by protamine, a heparin antidote. (2000) Mol. Biol. Cell. 11(7), 2213-2219.
116. Koulen P, Thrower E C. Pharmacological modulation of intracellular Ca(2+) channels at the single-channel level. Mol Neurobiol. 2001 August-December;24(1-3):65-86.
117. Koulen P, Janowitz T, Johenning F W, Ehrlich B E. Characterization of the calcium-release channel/ryanodine receptor from zebrafish skeletal muscle. J Membr Biol. 2001 Oct. 1; 183(3):155-63.
118. Ramos-Franco, J, M Fill, and G A Mignery. Isoform-specific function of single inositol 1,4,5-trisphosphate receptor channels. (1998) Biophys J. 75, 834-839.
119. Kaftan E J, Ehrlich B E, Watras J. Inositol 1,4,5-trisphosphate (InsP3) and calcium interact to increase the dynamic range of InsP3 receptor-dependent calcium signaling. J Gen Physiol. 110, 529-38, 1997.
120. Watras J, Bezprozvanny I, Ehrlich B E. Inositol 1,4,5-trisphosphate-gated channels in cerebellum: presence of multiple conductance states. (1991) J Neurosci. 11(10), 3239-3245.
121. Ramos-Franco J, Bare D, Caenepeel S, Nani A, Fill M, Mignery G. Single-channel function of recombinant type 2 inositol 1,4,5-trisphosphate receptor. (2000) Biophys J. 79(3), 1388-1399.
122. Michikawa T, Hirota J, Kawano S, Hiraoka M, Yamada M, Furuichi T, Mikoshiba K. Calmodulin mediates calcium-dependent inactivation of the cerebellar type 1 inositol 1,4,5-trisphosphate receptor. (1999) Neuron. 23(4), 799-808.
123. Koulen P, Kuhn R, Wassle H, Brandstatter J H. Modulation of the intracellular calcium concentration in photoreceptor terminals by a presynaptic metabotropic glutamate receptor. (1999) Proc Natl Acad Sci USA. 96(17), 9909-9914.
124. Friedrich W. Johenning, Michal Zochowski, Stuart J. Conway, Andrew B. Holmes, Peter Koulen, and Barbara E. Ehrlich. (2002) Distinct Intracellular Calcium Transients in Neurites and Somata Integrate Neuronal Signals J. Neurosci. 22(13), 5344-5353.
125. Khan Z U, Koulen P, Rubinstein M, Grandy D K, Goldman-Rakic P S. An astroglia-linked dopamine D2-receptor action in prefrontal cortex. (2001) Proc Natl Acad Sci USA. 98, 1964-1969.
126. Dubal D B, Zhu H, Yu J, Rau S W, Shughrue P J, Merchenthaler I, Kindy M S. Wise P M. Estrogen receptor alpha, not beta, is a critical link in estradiol-mediated protection against brain injury. Proc Natl Acad Sci USA. 2001; 98: 1952-1957.
127. Simpkins J W, Rajakumar G, Zhang Y Q, Simpkins C E, Greenwald D, Yu C J, Bodor N, Day A L. Estrogens may reduce mortality and ischemic damage caused by middle cerebral artery occlusion in the female rat. J Neurosurg. 1997; 87: 724-730.
128. Zhang Y Q, Shi J, Rajakumar G, Day A L, Simpkins J W. Effects of gender and estradiol treatment on focal brain ischemia. Brain Res. 784: 321-324, 1998.
129. Alkayed N J, Harukuni I, Kimes A S, London E D, Traystman R J, Hum P D. Gender-linked brain injury in experimental stroke. Stroke 29: 159-165, 1998.
130. Rusa R, Alkayed N J, Crain B J, Traystman R J, Kimes A S, London E D, Klaus J A. Hum P D. 17beta-estradiol reduces stroke injury in estrogen-deficient female animals. Stroke 30: 1665-1670, 1999.
131. Hum P D. Macrae I M. Estrogen as a neuroprotectant in stroke. J Cereb Blood Flow Metab 20:631-52, 2000.
132. Shi J, Bui J D, Yang S H, He Z, Lucas T H, Buckley D L, Blackband S J, King M A, Day A L. Simpkins J W. Estrogens decrease reperfusion-associated cortical ischemic damage: an MRI analysis in a transient focal ischemia model. Stroke 32: 987-992, 2001.
133. Sawada M, Alkayed N J, Goto S, Crain B J, Traystman R J, Shaivitz A, Nelson R J. Hum P D. Estrogen receptor antagonist ICI182,780 exacerbates ischemic injury in female mouse. J Cereb Blood Flow Metab 20: 112-118, 2000.
134. Sampei K, Goto S, Alkayed N J, Crain B J, Korach K S, Traystman R J, Demas G E, Nelson R J. Hum P D. Stroke in estrogen receptor-alpha-deficient mice. Stroke 31: 738-743, 2000.
135. Dubal D B, Kashon M L, Pettigrew L C, Ren J M, Finklestein S P, Rau S W, Wise P M. Estradiol protects against ischemic injury. J Cereb Blood Flow Metab. 18: 1253-1258, 1998.
136. Yang S H, Shi J, Day A L. Simpkins J W. Estradiol exerts neuroprotective effects when administered after ischemic insult. Stroke 31: 745-749, 2000.
137. He Z, He Y J, Day A L, Simpkins J W. Proestrus levels of estradiol during transient global cerebral ischemia improves the histological outcome of the hippocampal CAI region: perfusion-dependent and-independent mechanisms. J Neurol Sci. 193(2):79-87. 2002.
138. Yang S H, He Z, Wu S S, He Y J, Cutright J, Millard W J, Day A L. Simpkins J W. 17-beta estradiol can reduce secondary ischemic damage and mortality of subarachnoid hemorrhage. J Cereb Blood Flow Metab. 2001; 21: 174-181.
139. Hillard C. J., W. S. Edgemond, W. B. Campbell, Characterization of Ligand Binding to the Cannabinoid Receptor of Rat Brain Membranes using a Novel Method: Application to Anandamide, J. Neurochem. 64(2): 677-83 (1995).
140. Mak D O, Foskett J K. Single-channel kinetics, inactivation, and spatial distribution of inositol trisphosphate (IP3) receptors in Xenopus oocyte nucleus. (1997) J Gen Physiol. 109(5), 571-587.
141. Koulen, P. Chapter 26: Pharmacology of intracellular calcium channels, in: Planar Lipid Bilayers (BLMs) and their Applications, Ed. H T Tien and A. Ottova, Elsevier Science, New York, 2003.
142. Hingorani S R, Agnew W S. Assay and purification of neuronal receptors for inositol 1,4,5-trisphosphate. (1992) Methods Enzymol. 207, 573-591.
143. Raghupathi R, Graham D I, McIntosh T K. Apoptosis after traumatic brain injury. J Neurotrauma. (2000) 17(10):927-38.
144. Zipfel G J, Babcock D J, Lee J M, Choi D W. Neuronal apoptosis after CNS injury: the roles of glutamate and calcium. J Neurotrauma. (2000) October;17(10):857-69.
145. Fiskum G. Mitochondrial participation in ischemic and traumatic neural cell death. J Neurotrauma. (2000) 17(10):843-55.
146. Satoh T, Nakatsuka D, Watanabe Y, Nagata I, Kikuchi H, Namura S. Neuroprotection by MAPK/ERK kinase inhibition with U0126 against oxidative stress in a mouse neuronal cell line and rat primary cultured cortical neurons. Neurosci Lett. 2000 Jul. 14;288(2):163-6.
147. Desire L, Courtois Y, Jeanny J C., Endogenous and exogenous fibroblast growth factor 2 support survival of chick retinal neurons by control of neuronal neuronal bcl-x(L) and bcl-2 expression through a fibroblast berowth factor receptor 1- and ERK-dependent pathway. J Neurochem 75:151-63, 2000.
148. Wixler, V., Smola U., Schuler M. and Rapp U., Differential regulation of Raf isozymes by growth versus differentiation inducing factors in PC12 pheochromocytoma cells (1996) FEBS Lett 385:131-7
149. Gahwiler B (1981) Organotypic monolayer cultures of nervous tissue. J Neurosci Methods 4:329-342.
150. Schmid, HHO, Schmid, PC, and Berdyshev,EV (2002) Cell signaling by endocannabinoids and their cogeners: questions of selectivity and other challenges. Chem. Phys. Lipids 121: 111-134
151. Rosler, M The efficacy of cholinesterase inhibitors in treating the behavioural symptoms of dementia. Int J Clin Pract Suppl. 2002 127:20-36
152. McMurray R W and Hardy K J, Cox-2 inhibitors: today and tomorrow. Am J Med Sci. 2002 323:181-9
153. McGeer P L and McGeer E G Inflammation, autotoxicity and Alzheimer disease. Neurobiol Aging 2001 22(6):799-809
154. Moosmann B and Behl C; Antioxidants as treatment for neurodegenerative disorders. Expert Opin Investig Drugs. 2002 10:1407-35
155. Tang M X, Jacobs D, Stem Y, Marder K, Schofield P, Gurland B, Andrews H, Mayeux R. Effect of oestrogen during menopause on risk and age at onset of Alzheimer's disease. Lancet. 1996 348(9025):429-32
156. Takahashi M, Kohara A, Shishikura J I, Kawasaki-Yatsugi S, Ni J W, Yatsugi S I, Sakamoto S, Okada M, Shimizu-Sasamata M, Yamaguchi T. Y M872: A Selective, Potent and Highly Water-Soluble alpha-Amino-3-Hydroxy-5-Methylisoxazole-4-Propionic Acid Receptor Antagonist. CNS Drug Rev 2002 Winter;8(4):337-352
157. Green P S and Simpkins J W, Neuroprotective effects of estrogens: potential mechanisms of action. Int J Dev Neurosci. 2000 18(4-5):347-58
158. Brinton R D, Cellular and Molecular Mechanisms of Estrogen Regulation of Memory Function and Neuroprotection against Alzheimer's Disease: Recent Insights and Remaining Challenges, Learning and Memory 2001 8:121-133
159. Behl C Oestrogen as a neuroprotective hormone. Nat Rev Neurosci. 2002 6:433-42
160. Green P S and Simpkins J W, Neuroprotective effects of estrogens: potential mechanisms of action. Int J Dev Neurosci. 2000 18(4-5):347-58
161. Windisch M, Hutter-Paier B, Schreiner E. Current drugs and future hopes in the treatment of Alzheimer's disease. J Neural Transm Suppl 2002 62:149-64
162. Lee R, Kermani P, Teng K K, Hempstead B L. Regulation of cell survival by secreted proneurotrophins. Science. 2001 294(5548):1945-8
163. Green P S and Simpkins J W. Estrogens and estrogen-like non-feminizing compounds. Their role in the prevention and treatment of Alzheimer's disease. Ann N Y Acad Sci. 2000;924:93-8
164. Dhandapani K M, Brann D W. Protective effects of estrogen and selective estrogen receptor modulators in the brain. Biol Reprod. 2002 67(5):1379-85
165. Littleton-Kearney M T, Ostrowski N L, Cox D A, Rossberg M I, Hurn P D Selective estrogen receptor modulators: tissue actions and potential for CNS protection.CNS Drug Rev. 2002 8(3):309-30
166. Massa S M, Xie Y, Longo F M Alzheimer's therapeutics: neurotrophin small molecule mimetics. J Mol Neurosci. 2002 19(1-2):107-11
167. Leite, M. F., E. C. Thrower, W. Echevarria, P. Koulen, K. Hirata, A. M. Bennett, B. E. Ehrlich, and M. H. Nathanson. Nuclear and cytosolic calcium are regulated independently, PNAS. 100, 2975-80, 2003.
168. S.-Y. Hwang, J. Wei, J. H. Westhoff, R. S. Duncan, F. Ozawa, P. Volpe, K. Inokuchi, and P. Koulen. Differential functional interaction of two Vesl/Homer protein isoforms with ryanodine receptor type 1: A novel mechanism for control of intracellular calcium signaling. Cell Calcium, 34:177-84, 2003.
169. J. H. Westhoff, S.-Y. Hwang, R. S. Duncan, F. Ozawa, P. Volpe, K. Inokuchi, P. Koulen. Vesl/Homer proteins regulate ryanodine receptor type 2 function and intracellular calcium signaling. Cell Calcium, 34/3: 261-9, 2003.

1. A composition that provides neuroprotection by modulating intracellular calcium concentrations when administered to a subject, the composition comprising an effective amount of an N-acylethanolamine.

2. The composition of claim 1, further comprising a pharmaceutically acceptable carrier.

3. The composition of claim 1, wherein the effective amount of N-acylethanolamine is between about 0.01 and 500 mg/kg of the subject's weight.

4. The composition of claim 1, wherein the effective amount of N-acylethanolamine is between about 1 and 50 mg/kg of the subject's weight.

5. The composition of claim 1, wherein the N-acylethanolamine is selected from the group consisting of N-acylethanolamine 12:0, 14:0, 16:0, 18:0 and 18:2.

6. The composition of claim 1, wherein the N-acylethanolamine is plant-derived.

7. The composition of claim 1, wherein the N-acylethanolamine increases intracellular calcium release from intracellular stores.

8. The composition of claim 1, wherein the N-acylethanolamine decreases intracellular calcium release from intracellular stores.

9. The composition of claim 1, wherein the N-acylethanolamine crosses the blood-brain barrier.

10. The composition of claim 1, wherein the N-acylethanolamine is dissolved in a water, a saline or a lipophilic pharmacophor solution and is suitable for intravenous administration.

11. The composition of claim 1, wherein the N-acylethanolamine is dissolved in a lipophilic pharmacophor suitable for oral administration.

12. A method for treating neurodegenerative conditions, the method comprising the step of administering to a subject in need thereof a composition comprising an effective amount of an N-acylethanolamine.

13. The method of claim 12, further comprising a pharmaceutically acceptable carrier.

14. The method of claim 12, wherein the effective amount of N-acylethanolamine is between about 1 and 50 mg/kg of the subject's weight

15. The method of claim 12, wherein the effective amount of N-acylethanolamine is between about 1 and 10 mg/kg of the subject's weight.

16. The method of claim 12, wherein the N-acylethanolamine is selected from the group consisting of N-acylethanolamine 12:0, 14:0, 16:0, 18:0 and 18:2.

17. The method of claim 12, wherein the N-acylethanolamine is plant-derived

18. The method of claim 12, wherein the N-acylethanolamine increases intracellular calcium release from intracellular stores.

19. The method of claim 12, wherein the N-acylethanolamine decreases intracellular calcium release from intracellular stores.

20. The method of claim 12, wherein the N-acylethanolamine crosses the blood-brain barrier.

21. The method of claim 12, wherein the N-acylethanolamine is dissolved in a lipophilic pharmacophor and is suitable for intravenous injection.

22. The method of claim 12, wherein the N-acylethanolamine is dissolved in a lipophilic pharmacophor and is suitable for oral administration.

23. The method of claim 12, in which said administration of said composition is carried out over a period of at least about 3 days.

24. The method of claim 12, wherein said composition is administered one or more times daily over a predetermined period.

25. The method of claim 12, wherein the neurodegenerative conditions comprises ischemic cerebral trauma.

26. The method of claim 12, wherein the subject is human.

27. A method for treating ischemic cerebral trauma, the method comprising the step of administering to a subject in need thereof a composition comprising an effective amount of a plant-derived N-acylethanolamine.

28. The method of claim 27, wherein said composition is administered no later tan about 1, 4, 8, 24, or even 48 hours after the occurrence of said ischemic cerebral trauma.

29. A method for inhibiting apoptosis under ischemic conditions in an individual in need of such inhibition, the method comprising the step of administering to the individual an effective amount to inhibit apoptosis under ischemic conditions of a composition comprising at least one N-acylethanolamine and a pharmaceutically acceptable carrier.

30. A method for modulating the intracellular calcium concentration, comprising the step of administering to a cell an effective amount of at least one N-acylethanolamine.

31. A method of neuroprotection against ischemia, comprising administering to a subject an effective amount of at least one N-acylethanolamine to protect the cerebral cortex and the basal ganglia.

32. The method of claim 31, wherein ischemic injury is prevented and cannabinoid receptors are not activated.

33. A compound that provides neuroprotection comprising the following formula: 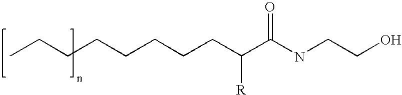
where: x is 1, 2, 3, 4, 5, 6 or more;
and R is an alkyl, an aminoethanol or an aminoalcohol; and enantiomers thereof.

34. A compound that provides neuroprotection comprising the following formula: 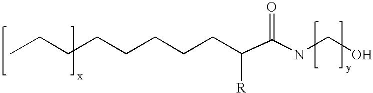
where: x is 1, 2, 3, 4, 5, 6;
where: y is 1, 2, 3, 4, 5, 6;
where R is an alkyl, an aminoethanol or an aminoalcohol; and enantiomers thereof.

35. A method for treating a condition in a subject, the method comprising the step of administering to a subject in need thereof a composition comprising an effective amount of a plant-derived N-acylethanolamine, wherein the conditions is selected from the group consisting of Alzheimer's disease, stroke, traumatic head and spinal cord injury, glaucoma, retinal ischemia, cardiac failure and ischemia and cancer.

36. The method of claim 35, wherein the NAEs is administered prior to, during, or after the observation of symptoms of diseases involving perturbation of the intracellular calcium homeostasis and to prevent the progression of the condition.

37. A method for modulating an intracellular calcium channel of a neuronal cell in a host comprising determining the level of intracellular calcium channel signaling in the host and administering to the host a formulation containing an NAE, only if the level of signaling needs modulation.

38. The method of claim 37, wherein the level of intracellular calcium channel signaling is determined is suspected of having Alzheimer's disease, stroke, traumatic head and spinal cord injury, glaucoma, retinal ischemia, cardiac failure and ischemia and cancer.

39. The method of claim 37, wherein the effective amount of N-acylethanolamine is between about 1 and 50 mg/kg of the subject's weight.

40. The method of claim 37, wherein the effective amount of N-acylethanolamine is between about 0.01 and 500 mg/kg of the subject's weight.

41. The method of claim 37, wherein the N-acylethanolamine is selected from the group consisting of N-acylethanolamine 12:0, 14:0, 16:0, 18:0 and 18:2.

42. The method of claim 37, wherein the N-acylethanolamine is isolated and purified from a plant.

43. The method of claim 37, wherein the N-acylethanolamine is isolated and purified from a plant-derived extract.

44. The method of claim 37, wherein the N-acylethanolamine is plant-derived and provided as a nutritional supplement.

45. The method of claim 37, wherein the N-acylethanolamine increases intracellular calcium release from intracellular stores.

46. The method of claim 37, wherein the N-acylethanolamine decreases intracellular calcium release from intracellular stores.

47. The method of claim 37, wherein the N-acylethanolamine crosses the blood-brain barrier.
